(19) United States
(12) Patent Application Publication
(10) Pub. No.: US 2022/0062205 A1
(43) Pub. Date: Mar. 3, 2022

(54) TREATMENT OF GASTROINTESTINAL DISORDERS AND SYMPTOMS THEREOF

(71) Applicant: Indiana University Research and Technology Corporation, Indianapolis, IN (US)

(72) Inventors: Mark R. Kelley, Zionsville, IN (US); Kulmira Nurgali, Victoria (AU)

(21) Appl. No.: 17/415,065

(22) PCT Filed: Dec. 11, 2019
(86) PCT No.: PCT/US19/65624

§ 371 Date: Jun. 17, 2021

Related U.S. Application Data
(60) Provisional application No. 62/780,574, filed on Dec. 17, 2018.
(60) Provisional application No. 62/862,808, filed on Jun. 18, 2019.

(51) Int. Cl. A61K 31/165; A61K 45/06; A61P 29/00

(52) U.S. Cl. CPC A61K 31/165; A61P 29/00; A61K 45/06

(57) ABSTRACT

Methods of reducing inflammation and chronic pain in the gut of a subject suffering from inflammatory bowel disease (IBD) are disclosed herein. Particularly disclosed are methods of administrating the apurinic/apyrimidinic endonuclease 1 redox factor 1 (APE1/Ref-1) inhibitor, APX3330, which blocks APE1 and regulates transcription factors (TFs) involved in inflammation, thereby alleviating inflammatory or chronic pain.

### CROSS-REFERENCE TO RELATED APPLICATIONS

The present application claims priority to U.S. Provisional Application No. 62/780,574, filed Dec. 17, 2018 and to U.S. Provisional Application No. 62/862,808, filed Jun. 18, 2019, both of which are incorporated by reference in their entireties.

### BACKGROUND OF THE DISCLOSURE

The present disclosure relates generally to methods of reducing neuronal sensitivity, thereby reducing inflammation and chronic pain in the gut. Particularly, it has been found herein that by blocking the APE1 pathway, through the administration of an apurinic/apyrimidinic endonuclease 1 redox factor 1 (APE1/Ref-1) inhibitor (e.g., APX3330), transcription factors (TFs) involved in inflammation of the gastrointestinal tract are regulated, thereby alleviating inflammatory or chronic pain in the gut of subjects suffering from gastrointestinal disorders, and in particular, inflammatory bowel disease (IBD).

The enteric nervous system (ENS) controls or regulates vital gastrointestinal functions, including motility, secretion, local immunity, and inflammation, and represents the largest collection of autonomous neurons outside of the brain. Disorders involving the ENS (e.g., inflammatory bowel disease (IBD)) are common and major contributors to the health burden throughout the world.

Inflammatory bowel disease (IBD), associated with damage to the ENS, is characterized by chronic severe inflammation of the small bowel and/or the colon leading to recurrent diarrhea and abdominal pain. Crohn disease (CD) and ulcerative colitis (UC) are the two main clinicopathological subtypes of IBD. Despite both being chronic and relapsing inflammatory diseases of the bowel, they can be differentiated by the location of the inflammation in the gastrointestinal tract and by the nature of the histological alterations in the intestinal wall. Anatomically, CD can affect the entire gastrointestinal tract from mouth to anus, although it commonly affects the terminal ileum and colon. UC is restricted to the rectum, colon and caecum. Microscopically, CD is transmural and often discontinuous while UC affects only the intestinal mucosa in a continuous pattern.

IBD is a very disabling disease due to the fatigue associated with the inflammatory symptoms and due to the chronic pain suffered by patients. Approximately, 1.6 million Americans currently have IBD, a growth of about 200,000 since 2011. The pathogenesis of IBD is only partially understood; various environmental and host (e.g., genetic, epithelial, immune and nonimmune) factors are involved. Complex interactions between the immune system, enteric commensal bacteria/pathogens and host genotype are thought to underlie the development of IBD. These relapsing chronic inflammatory disorders appear to be caused by overly aggressive T-cell responses directed against environmental factors and/or a subset of commensal bacteria/pathogens that inhabit the distal ileum and colon of genetically susceptible hosts.

In IBD, the existence of a genetic vulnerability leads to disrupted identification and presentation of intestinal antigens to effector cells. The subsequent inappropriate adaptive immune response results in loss of tolerance to commensal flora and to amplification and maintenance of the inflammatory response to intestinal pathogens, especially in CD where there is a weakness of the immune system. In parallel to inflammation, infiltration of immune cells in the intestinal mucosa and in the proximity of nerve endings leads to enteric neuro-immune direct contacts. These interactions cause the activation of visceral afferents which is the first step to the development of chronic abdominal pain consecutive to inflammation.

Currently, there is no cure or effective treatment for patients diagnosed with functional gastrointestinal diseases such as IBD. The main goal of current therapies for IBD is to induce a clinical remission by focusing on symptoms and then maintain it for a long period of time, in order to realize the best attainable quality of life. As current therapies have limited efficacy, new therapies for treating inflammatory and chronic pain in the gut of subjects with IBD is clinically significant. Accordingly, the present disclosure provides insight into the pathway to alleviate inflammation and/or chronic pain. Further, the present disclosure provides a compound, APX3330, to reduce neuronal sensitivity and oxidative stress, thereby reducing inflammation and chronic pain in the gut.

### BRIEF DESCRIPTION

The present disclosure relates generally to methods of regulating transcription factors (TFs) involved in gut inflammation, thereby reducing inflammatory and chronic pain in the gut of subjects suffering from gastrointestinal diseases and particularly disorders such as IBD. Particularly, it has been found herein that by blocking APE1, through the administration of APX3330 (and/or analogs thereof), TFs such as STATS, AP-1, NFκB and the like under APE1 redox control are regulated, thus reducing neuronal sensitivity to inflammatory mediators and alleviating inflammation or chronic pain in the gut of subjects suffering from gastrointestinal disorders (e.g., inflammation of the gastrointestinal (GI) track, irritating bowel, indeterminate colitis (IC), functional GI disease, inflammatory bowel disease (IBD), and effects on the enteric nervous system (ENS)). Furthermore, these GI disorders have been known as precursors to colorectal cancer (CRC) such that APX3330 will not only alleviate the GI disorder, but be preventive for CRC.

Further, oxidative stress plays an important role in pathophysiological mechanisms involved in inflammation induced enteric neuronal loss and damage (i.e., enteric neuropathy). Apurinic/Apyrimidinic Endonuclease 1/Redox Factor-1 (APE1/Ref-1) is a vital dual functioning protein that acts as an essential regulator of cellular responses to oxidative stress.

Based on the foregoing, in one aspect, the present disclosure is directed to a method of treating inflammation and chronic pain in a subject suffering from functional gastrointestinal disease, the method comprising administering to the subject an effective amount of an apurinic/apyrimidinic endonuclease 1 redox factor 1 (APE1/Ref-1) inhibitor, pharmaceutically acceptable salts or pharmaceutically acceptable solvates thereof.

In another aspect, the present disclosure is directed to a method of reducing neuronal loss in a subject suffereing from functional gastrointestinal disease, the method comprising administering to the subject an effective amount of an apurinic/apyrimidinic endonuclease 1 redox factor 1 (APE1/Ref-1) inhibitor, pharmaceutically acceptable salts or pharmaceutically acceptable solvates thereof.

In yet another aspect, the present disclosure is directed to a method of enhancing neurogenesis in a subject suffering from functional gastrointestinal disease, the method comprising administering to the subject an effective amount of an apurinic/apyrimidinic endonuclease 1 redox factor 1 (APE1/Ref-1) inhibitor, pharmaceutically acceptable salts or pharmaceutically acceptable solvates thereof, which selectively inhibits the amino terminal portion of APE1.

In yet another aspect, the present disclosure is directed to a method of myenteric and enteric neuronal protection in a subject in need thereof, the method comprising administering to the subject an effective amount of an apurinic/apyrimidinic endonuclease 1 redox factor 1 (APE1/Ref-1) inhibitor, pharmaceutically acceptable salts or pharmaceutically acceptable solvates thereof.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIGS. 1A-1C shows disease activity in Winnie mice, which are used as a murine model of IBD. FIG. 1A depicts body weight loss or gain relative to initial weight prior to treatment in control, Winnie-Sham and Winnie-APX3330 treated mice. FIG. 1B depicts the severity of intestinal inflammation indicated by presence of rectal prolapse with blood vessel proliferation and oedema Images taken at day 14 of treatment in control, Winnie-Sham and Winnie-APX3330 treated mice. FIG. 1C depicts faecal water content following repeated treatments over the 14-day period. Wet weight of fresh faecal pellets was measured immediately upon pellet expulsion. Pellets were than dehydrated over night at room temperature and the dry weight was measured. The faecal water content was calculated as the difference between the wet and dry weight.

FIGS. 2A-2E show gastrointestinal transit of barium sulfate as analyzed in the Example. FIG. 2A depicts gastrointestinal transit time in control, Winnie-Sham, and Winnie-APX3330, treated mice measured by in vivo X-ray imaging obtained every 5 minutes for the first hour, every 10 minutes for the second hour and every 20 minutes for the last hour after intragastric administration of barium sulfate by oral gavage. X-ray imaging was ceased once the mice had expulsed a pellet containing barium sulfate. FIG. 2B depicts transit time (min) for barium sulfate moving from the stomach to the caecum (oro-caecal transit time (OCTT)). FIG. 2C depicts transit time (min) for barium sulfate moving from the cecum to anus (colonic transit time (CTT)). FIG. 2D depicts cecum retention time calculated by the difference between CTT and OCTT. FIG. 2E depicts the total transit time calculated as time from intragastric administration of barium sulfate till the expulsion of a pellet containing barium sulfate.

FIGS. 3A-3D shows colonic contractile activity in Winnie mice as analyzed in the Example Ex-vivo analysis of colonic motility in excised whole colons from control, Winnie-Sham and Winnie-APX3330 treated mice. FIG. 3A depicts examples of spatiotemporal maps depicting colonic contractions (red channel) and relaxations (blue channel) of whole length colons. FIG. 3B depicts the length of colonic contractions relative to the total length of the colon.

FIG. 3C depicts the length of short contractions (<50% of colon length) relative to the total length of the colon. FIG. 3D depicts the length of colonic migrating motor complexes (>50% of colon length) relative to the total length of the colon.

FIGS. 4A-4C show colonic smooth muscle cells in Winnie mice as analyzed in the Example. FIG. 4A depicts colon cross sections from control, Winnie-Sham and Winnie-APX3330 treated mice immunolabelled by anti-smooth muscle actin (α-SMA) antibody counterstained with DAPI. Smooth muscle cells were analysed within the circular muscle layer of the colon. Scale bar=50 μm. FIG. 4B depicts that the size of α-SMA-IR cells observed deceased in Winnie-sham treated (n=5) compared to both control C57BL/6 (n=5) and Winnie-APX3330 treated (n=5) mice. FIG. 4C depicts α-SMA-IR cell number within the circular muscle counted in control C57BL/6 (n=5), Winnie-sham treated (n=5) and Winnie-APX3330 treated (n=5) mice. Data expressed as mean±SEM, **P<0.01, ****P<0.0001 when compared to control C57BL/6; \hat{ }\hat{ }\hat{ } \hat{ }P<0.0001 when compared to Winnie-sham treated mice.

FIGS. 5A & 5B show that APX3330 treatment ameliorated nerve fiber density in Winnie mice. FIG. 5A shows neuronal microtubule proteins stained by immunofluorescence using the marker β-tubulin (III) (purple channel) anti-body to identify nerve fiber processing innervating the colon in cross sections. FIG. 5B depicts the density of β-tubulin (III)-IR nerve fibers normalised to colon thickness in control C57BL/6 (n=9), Winnie-sham treated (n=8) and Winnie-APX3330 treated (n=5) mice. Data expressed as mean±SEM, ****P<0.0001, when compared to control C57BL/6: \hat{ }\hat{ }\hat{ }P<0.001 when compared to Winnie-sham treated mice.

FIGS. 6A-6D depict morphological changes in Winnie mice as analyzed in the Example. FIG. 6A depicts gross morphological changes analysed by H&E staining in colon cross sections from control, Winnie-Sham and Winnie-APX3330 treated mice. FIG. 6B depicts Goblet cell density analysed by Alcian Blue staining in colon cross sections from control, Winnie-Sham and Winnie-APX3330 treated mice. FIG. 6C depicts histological scoring of morphological damage to the colon based on the following parameters: changes in crypt architecture (0-5), reduction in crypt length (0-5), mucosal ulceration (0-5), and immune cell infiltration (0-5) (total score 20). FIG. 6D depicts Goblet cell density determined in the distal colon mucosa of cross sections. Data expressed as mean±SEM, **P<0.014, ***P<0.001, ****P<0.0001, when compared to control C57BL/6; \hat{ }\hat{ }P<0.01, \hat{ }\hat{ }\hat{ }\hat{ }P<0.0001 when compared to Winnie-sham treated mice.

FIGS. 7A & 7B show intestinal permeability and inflammation in Winnie mice as analyzed in the Example FIG. 7A depicts intestinal permeability measured by the level of fatty acid-binding protein 1 (FABP1) in the blood serum at day 14 for all treatment groups.

FIG. 7B is an assessment of colonic inflammation via measurement of faecal Lipocalin (Lcn)-2 levels at day 14 in faecal samples from control, Winnie-Sham and Winnie-APX3330 treated mice.

FIGS. 8A & 8B depict the effects of APX3330 treatment on glial cell density in the myenteric plexus of Winnie mice. FIG. 8A depicts glial cells stained with an immunofluorescence marker against GFAP (orange channel) in the myenteric plexus from control C57BL/6, Winnie-sham treated and Winnie-APX3330 treated mice. (Scale bar=100 μm). FIG. 8B depicts density of GFAP-IR glial cells relative to ganglia size in control C57BL/6 (n=5), Winnie-sham treated (n=5) and Winnie-APX3330 treated mice (n=4). Data expressed as mean±SEM, **P<0.01, when compared to control C57BL/6: \hat{ }P<0.05 when compared to Winnie-sham treated mice.

FIGS. 9A & 9B show myenteric neurons from Winnie mice as analyzed in the Example. FIG. 9A depicts immunolabelling of myenteric neurons by anti-microtubule associated protein 2 (MAP2) antibody in colon wholemount preparations exposing the myenteric ganglia from control, Winnie-Sham and Winnie-APX3330 treated mice. Scale bar=50 μm. FIG. 9B depicts MAP2-immunoreactive neurons in the myenteric ganglia.

FIGS. 10A & 10B depict APX3330 treatment on superoxide production in the myenteric plexus of Winnie mice. FIG. 10A shows that LMMP preparations of the distal colon were stained with immunofluorescence marker MitoSOX (red channel) indicative of oxidative stress. Increased expression for Mitosox was evident in the ganglia of Winnie-sham treated (n=4) compared to control C57BL/6 (n=4). This was alleviated in Winnie-APX3330 treated (n=4) mice (scale bar=100 μm) FIG. 10B depicts quantification of MitoSOX fluorescence intensity assessed relative to ganglion area. Data expressed as mean±SEM, ****P<0.0001, when compared to control C57BL/6: \hat{ }\hat{ }\hat{ }\hat{ }P<0.0001 when compared to Winnie-sham treated mice.

FIGS. 11A & 11B depict APX3330 treatment on HMGB1 translocation in the myenteric plexus of Winnie mice. FIG. 11A depicts HMGB1-IR cells (green channel) colocalised with DAPI (blue channel) in the myenteric plexus. Retniret Translocation of nuclear HMGB1-IR to cytosol was observed in Winnie-sham treated (n=4) compared to control C57BL/6 (n=5). Translocation of HMGB1 was averted in Winnie-APX3330 treated mice (n=4) (scale bar=100 μm). FIG. 11B depicts translocation of HMBG1-IR cells quantified in the myenteric ganglia. Data expressed as mean±SEM, **P<0.01, when compared to control C57BL/6: \hat{ }\hat{ }P<0.01 when compared to Winnie-sham treated mice.

FIGS. 12A-12C show the expression of APE1 in the myenteric plexus of Winnie mice. FIG. 12A depicts Apurinic/apyrimidinic Endonuclease (APE1) immunoreactivity within the myenteric plexus in colon wholemount preparations from control, Winnie-Sham and Winnie-APX3330 treated mice. Scale bar=50 μm. FIG. 12B depicts the density of APE1-immunoreactivity within myenteric ganglia analysed by Image J software. FIG. 12C depicts the proportion of APE1-immunoreactive neurons within myenteric ganglia.

FIGS. 13A-13C show DNA damage in myenteric neurons of Winnie mice as analyzed in the Example. FIG. 11A depicts co-immunolabelling of myenteric neurons by anti-microtubule associated protein 2 (MAP2) antibody and anti-DNA damage (Oxo-8-dG ((8-Oxo-7,8-dihydro-2′-deoxyguanosine)) antibody in colon wholemount preparations exposing the myenteric ganglia from control, Winnie-Sham and Winnie-APX3330 treated mice. Scale bar=50 μm. FIG. 11B depicts the density of Oxo-8-dG-immunoreactivity within myenteric ganglia analysed by Image J software. FIG. 11C depicts the proportion of neurons with DNA damage relative to the total number of MAP2-immunoreactive neurons in the myenteric ganglia.

FIGS. 14A & 14B depict the effects of APX3330 treatment on inflammatory and oxidative gene regulators in the distal colon of Winnie mice. Particularly, FIGS. 14A & 14B show RNA sequencing of pooled distal colon (control C57BL/6, n=6; Winnie-sham treated, n=6 and Winnie-APX3330, n=6) samples determined the efficacy of APX3330 treatment on upregulated expression for inflammatory and oxidative stress markers. Winnie-sham treated animals measured increased fold change for S100a8, Khdc1a, Lrg1, Retnlb, Nos2, Ido1 and REG3B. APX3330 treatment in Winnie mice caused a significant (fold change>±2) down regulation for these RNA expressions relative to control C57BL/6 mice.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

The present disclosure relates generally to methods of reducing inflammatory and chronic pain in the gut of subjects suffering from functional gastrointestinal diseases and particularly disorders such as IBD. Particularly, it has been found herein that by blocking APE1, through the administration of APX3330 (and/or analogs thereof), transcription factors (TFs) involved in inflammation are regulated, thereby alleviating inflammation or chronic pain in the gut. Further, as APE1/Ref-1 is a dual functioning protein that acts as an essential regulator of cellular responses to oxidative stress, which stress plays an important role in pathophysiological mechanisms involved in inflammation induced enteric neuronal loss and damage, blocking APE1 through administration of APX3330 further reduces oxidative stress, thereby further reducing inflammation and chronic pain.

More particularly, the ENS is a division of the autonomic nervous system with intrinsic enteric neurons that control the gastrointestinal (GI) functions without assistance from the central nervous system. The ENS is comprised of an estimated 200-600 million neurons, which is equivalent to the spinal cord. The ENS encompasses a complex network of neurons and glial cells residing along the GI tract within the enteric ganglia forming two distinct plexi: the submucosal plexus and the myenteric plexus. Myenteric neurons are involved predominately in coordination of motility function, whereas submucosal neurons predominantly control secretion of endocrine and exocrine hormones involved in blood flow and absorption.

GI function is homeostatically maintained by the ENS. Damage to the ENS associates with GI dysfunction. In experimental animal models with GI inflammation it has been demonstrated that enteric neuropathy, morphological damage to neurons and enteric hyper-excitability occur. Myenteric and submucosal plexitis (inflammation in the plexi) in intestinal tissues resected from IBD patients has been implemented to predict post-operative reoccurrence of the disease.

The role of the ENS in GI immunity has strengthened as connections between enteric neurons and immune cells are correlated in both normal and pathological conditions. Enteric neurons and immune cells interact over the production and release of immune and neural mediators. Enteric nerve fibres form a connection within the lymphoid tissue and immune cells located inside the multiple layers along the GI tract, establishing a functional connection. Enteric glial cells produce both cytokines and neurotransmitters functioning to form neuroimmune interaction through cytokine receptors. Enteric neurons display receptors for soluble immune mediators consisting of cytokines and chemokines, in comparison, immune cells have receptors for neuropeptides.

Furthermore, enteric neurons have been shown to produce pro-inflammatory cytokines including interleukin-8 (IL-8). Neuronal electrophysiological activity driven by inflammatory cytokines alter GI motility and neural controlled secretory functions, as they are susceptible to compromised regulation via immune and neuroimmune interactions. The understanding of neuroimmune interactions in inflammatory conditions is critical to prolonging remission, enabling the ENS as an ideal target for the development of future therapies.

Intestinal inflammation-induced ENS damage is associated with a compromised GI antioxidant capacity. Studies have shown oxidative stress induced by chemotherapy and diabetes leads to enteric neuropathy. Investigating oxidative stress in inflammatory conditions will encompass insight to the pathogenesis of IBD and therapies.

APE1/Ref-1 acts as a dual functioning molecule containing a redox active domain and a DNA repair domain APE1/Ref-1 redox active domain regulates cellular stress responses, angiogenesis, inflammation, and proliferation. In oxidative stress, levels of NO and cellular differentiation are controlled by APE1/Ref-1, by subsiding proapoptotic tumour necrosis factor-α (TNF-α) signalling via pro-survival signaling.

In suitable embodiments, the present disclosure includes administering to a subject in need thereof an effective amount of an APE1 inhibitor, pharmaceutically acceptable salts or pharmaceutically acceptable solvates thereof, the APE1 inhibitor capable of interacting with the APE1 protein such to cause unfolding of the APE1 protein in the amino terminal portion of APE1, inhibiting the ability of APE1 to interact with other proteins in the neurons or to perform its redox signaling function. More particularly, APE1 inhibitors used in the present disclosure have anti-inflammatory effects, blocking the ability of APE1/Ref-1 to convert NF-κB and AP-1 from an oxidised state to reduced state, thereby altering their transcriptional activity. These inhibitors have been shown to suppress the production of pro-inflammatory cytokines and inflammatory mediators in murine macrophages. This results in the inability of NF-κB and AP-1 to bind to their target DNA sequence. Moreover, the inhibition allows direct down regulation of inflammatory cytokine secretion and ROS activation.

Targeting the specific inhibition of APE1/Ref-1 redox pathways and utilising the DNA repair domain can lead to a possible IBD and enteric neuropathy therapy, as studies have shown reverse of inflammation-induced changes in neuronal sensitivity.

Accordingly, in particular suitable embodiments, the APE1 inhibitor has the formula:

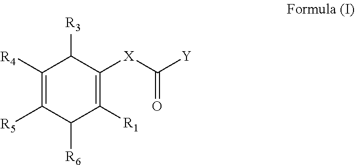

wherein R₁ is selected from the group consisting of alkyl, alkoxy, hydroxyl, and hydrogen; R₃ and R₆ are independently selected from the group consisting of a substituted or unsubstituted alkoxy, a substituted or unsubstituted aryl and an oxo; R₄ and R₅ are independently selected from the group consisting of an alkoxy and aryl, or both R₄ and R₅ taken together form a substituted or unsubstituted napthoquinone;

X is selected from the group consisting of CH═CR₂ and NCH, wherein R₂ is selected from the group consisting of C₁-C₁₀ alkyl and CF₃CH₂CH₂; and

Y is selected from the group consisting of N(Rz)R2 or NR\hat{ }OR\hat{ }, wherein each Rz is independently selected from the group consisting of C1-C6 alkyl, heteroalkyl, cycloalkyl and cycloheteroalkyl, straight or branched chain or optionally substituted, or both Rz and R2 taken together with the attached nitrogen form an optionally substituted heterocycle; where each R\hat{ } is independently selected from the group consisting of hydrogen, alkyl, heteroalkyl, cyclohexyl, and cycloheteroalkyl, each of which is optionally substituted, or both R\hat{ } are taken together with the attached nitrogen and oxygen to form an optionally substituted heterocycle.

Particularly suitable APE1 inhibitors include 3-[(5-(2,3-dimethoxy-6-methyl1,4-benzoquinoyl)]-2-nonyl-2-proprionic acid, (hereinafter “E3330” or “3330” or “APX3330”), and/or its analogs (e.g., [(2E)-2-[(3-methoxy-1,4-dioxo-1,4-dihydronaphthalen-2-yl)methylidene]-N,N-diethylpentanamide] (hereinafter “APX2009”), (2E)-2-[(3-methoxy-1,4-dioxo-1,4-dihydronapthalen-2-yl)methylidene]-N,N-dimethylpentanamide] (hereinafter “APX2007”), (2E)-2-[(3-methoxy-1,4-dioxo-1,4-dihydronapthalen-2-yl)methylidene]-N-methoxypentanamide] (hereinafter “APX2014”), (2E)-2-(3-methoxy-1,4-dioxo-1,4-dihydronaphthalen-2-yl)-N,N,2-trimethylprop-2-enamide (hereinafter “APX2032”)). Additional suitable analogs are shown below and in Table 1. Further information on APX3330 may be found in Abe et al., U.S. Pat. No. 5,210,239, and information on APX2009 may be found in Kelley et al., J Pharmacol Exp Ther. 2016 November, 359(2): 300-309, each incorporated herein by reference to the extent they are consistent herewith.

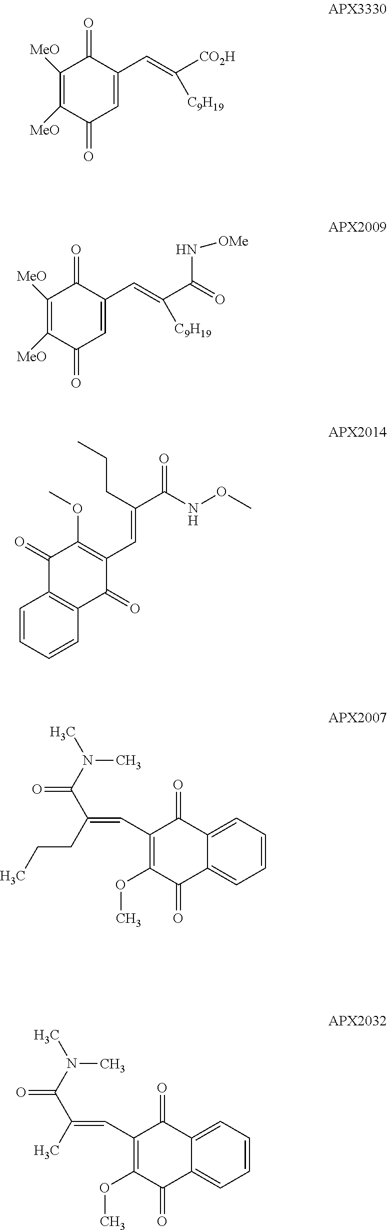

TABLE 1

| COMPOUND | | | | | | | | | | |
|---|---|---|---|---|---|---|---|---|---|---|
| ID | R₁ | X | C (═O)Y | R₂ | R₃ | R₄ | R₅ | R₆ | EF | MW |
| APX3330 | CH₃ | CH═CR₂ | OH | C₉H₁₉ | ═O | MeO | MeO | ═O | C₂₁H₃₀O₆ | 378.459 |
| APX2006 | MeO | CH═CR₂ | NMe | C₃H₇ | ═O | napthoquinone | | ═O | C₁₈H₁₉NO₄ | 313.353 |
| APX2007 | MeO | CH═CR₂ | N(Me)₂ | C₃H₇ | ═O | napthoquinone | | ═O | C₁₉H₂₁NO₄ | 327.38 |
| APX2008 | MeO | CH═CR₂ | NEt | C₃H₇ | ═O | napthoquinone | | ═O | C₁₉H₂₁NO₄ | 327.38 |
| APX2009 | MeO | CH═CR₂ | N(Et)2 | C₃H₇ | ═O | napthoquinone | | ═O | C₂₁H₂₅NO₄ | 355.428 |
| APX2010 | CH₃ | CH═CR₂ | NCH3 | C₄H₉ | ═O | napthoquinone | | ═O | C₁₇H₂₃NO₅ | 321.373 |
| APX2011 | CH₃ | CH═CR₂ | N(CH3)₂ | C₄H₉ | ═O | napthoquinone | | ═O | C₂₀H₂₃NO₃ | 325.408 |
| APX2012 | CH₃ | CH═CR₂ | NCH₂CH₃ | C₄H₉ | ═O | napthoquinone | | ═O | C₂₀H₂₃NO₃ | 325.408 |
| APX2013 | CH₃ | CH═CR₂ | N(Et)₂ | C₄H₉ | ═O | napthoquinone | | ═O | C₂₂H₂₇NO₃ | 353.462 |
| APX2014 | MeO | CH═CR₂ | NOMe | C₃H₇ | ═O | napthoquinone | | ═O | C₁₈H₁₉NO₅ | 329.352 |
| APX2015 | CH₃ | CH═CR₂ | N-cPro | C₄H₉ | ═O | napthoquinone | | ═O | C₂₁H₂₃NO₃ | 337.419 |
| APX2016 | CH₃ | CH═CR₂ | NOMe | C₄H₉ | ═O | napthoquinone | | ═O | C₁₉H₂₁NO₄ | 327.38 |
| APX2017 | CH₃ | CH═CR₂ | N-Et-Pip | C₄H₉ | ═O | napthoquinone | | ═O | C₂₄H₃₀N₂O₃ | 394.515 |
| APX2018 | CH₃ | CH═CR₂ | N-cHexyl | C₄H₉ | ═O | napthoquinone | | ═O | C₂₄H₂₉NO₃ | 379.492 |
| APX2019 | CH₃ | CH═CR₂ | 2-Piperdone | C₄H₉ | ═O | napthoquinone | | ═O | C₂₂H₂₄N₂O₄ | 380.444 |
| APX2020 | CH₃ | CH═CR₂ | N(Me)OMe | C₄H₉ | ═O | napthoquinone | | ═O | C₂₀H₂₃NO₄ | 341.407 |
| APX2021 | CH₃ | CH═CR₂ | E-Morpholino | C₄H₉ | ═O | napthoquinone | | ═O | C₂₂H₂₅NO₄ | 367.445 |
| APX2022 | CH₃ | CH═CR₂ | Z-Morpholino | C₄H₉ | ═O | napthoquinone | | ═O | C₂₂H₂₅NO₄ | 367.445 |
| APX2023 | CH₃ | CH═CR₂ | NH₂ | C₄H₉ | ═O | napthoquinone | | ═O | C₁₈H₁₉NO₃ | 297.348 |
| APX2024 | CH₃ | CH═CR₂ | E-NCH₂CH₂OMe | C₄H₉ | ═O | napthoquinone | | ═O | C₂₁H₂₅NO₄ | 355.434 |
| APX2025 | CH₃ | CH═CR₂ | Z-NCH₂CH₂OMe | C₄H₉ | ═O | napthoquinone | | ═O | C₂₁H₂₅NO₄ | 355.434 |
| APX2026 | Cl | CH═CR₂ | NOMe | C₃H₇ | ═O | napthoquinone | | ═O | C₁₇H₁₆ClNO₄ | 333.77 |
| APX2027 | Cl | CH═CR₂ | N(Et)₂ | C₃H₇ | ═O | napthoquinone | | ═O | C₂₀H₂₂ClNO₃ | 359.85 |
| APX2028 | OH | CH═CR2 | OH | C3H7 | ═O | napthoquinone | | ═O | C16H14O5 | 286.283 |
| APX2029 | MeO | CH═CR₂ | N(Et)₂ | C₃H₇ | ═O | napthoquinone | | ═O | C₂₁H₂₅N0₄ | 355.434 |
| APX2030 | Me | CH═CR₂ | N(Me)₂ | C₃H₇ | ═O | napthoquinone | | ═O | C₁₉H₂₁NO₃ | 311.381 |
| APX2031 | MeO | CH═CR₂ | NCH₃ | CH₃ | ═O | napthoquinone | | ═O | C₁₆H₁₅NO₄ | 285.295 |
| APX2032 | MeO | CH═CR₂ | N(CH₃)₂ | CH₃ | ═O | napthoquinone | | ═O | C₁₇H₁₇NO₄ | 299.321 |
| APX2033 | MeO | CH═CR₂ | OH | CH₃ | ═O | napthoquinone | | ═O | C₁₅H₁₂O₅ | 272.253 |
| APX2034 | MeO | CH═CR₂ | OH | C₃H₇ | ═O | napthoquinone | | ═O | C₁₇H₁₆O₅ | 300.306 |
| APX2043 | MeO | CH═CR₂ | N(CH₃)₂ | C₃H₇ | OH | napthoquinone | | OH | C₁₉H₂₅NO₄ | 331.412 |
| APX2044 | CF₃O | CH═CR₂ | N(Et)₂ | C₃H₇ | ═O | napthoquinone | | ═O | C₂₁H₂₂F₃NO₄ | 409.405 |
| APX2045 | CH₃ | CH═CR₂ | N(Et)₂ | C₃H₇ | ═O | napthoquinone | | ═O | C₂₁H₂₅NO₃ | 339.435 |
| APX2046 | CH₃ | CH═CR₂ | N(Et)₂ | CF₃CH₂CH₂ | ═O | napthoquinone | | ═O | C₂₁H₂₂F₃NO₃ | 393.406 |
| APX2047 | CH₃ | CH═CR₂ | N(Et)₂ | C₃H₇ | OCH₃ | napthoquinone | | OCH₃ | C₂₃H₃₁NO₃ | 369.505 |
| APX2048 | CH₃ | CH═CR₂ | NOCH₃ | C₉H₁₉ | ═O | MeO | MeO | ═O | C₂₃H₃₁NO₄ | 397.515 |
| APX2049 | CH₃ | CH═CR₂ | N(CH₃)CC(O)C(O)C(O)C(O)COH | C₉H₁₉ | ═O | MeO | MeO | ═O | C₂₈H₄₅NO₁₀ | 555.665 |
| APX2050 | CH₃ | CH═CR₂ | N(CH₃)OCH₃ | C₉H₁₉ | ═O | MeO | MeO | ═O | C₂₃H₃₅NO₆ | 421.534 |

It has herein been found that the administration of APX3330 (and/or its analogs) inhibits APE1 protein from interacting with other proteins in the neurons. This interaction inhibition blocks the activation of the transcription factors (TFs) through a reduction-oxidation mechanism Blocking of the TF activation results in the lack of their functional activity involving binding to the promoter region of genes involved in inflammation. Further, the inhibition allows for APE1 to be free to perform enhanced DNA repair functions at an oxidized or abasic site in damaged DNA (damaged by inflammatory and other effectors of neuronal pain pathway induction), thereby repairing the DNA and allowing for the proper activity of the genes needed for normal cellular function. Therefore, the mechanism is two-fold; blocking inflammatory TFs from being active as well as enhancing the repair of damaged DNA leading to the proper function of neuronal cells of the enteric nervous system (ENS), which controls vital gastrointestinal functions, e.g., local immunity and inflammation as well as pain.

Suitable dosages of the APE1 inhibitor, pharmaceutically acceptable salts or pharmaceutically acceptable solvates thereof, for use in the methods of the present disclosure will depend upon a number of factors including, for example, age and weight of an individual, severity of inflammatory or chronic pain, nature of a composition, route of administration and combinations thereof. Ultimately, a suitable dosage can be readily determined by one skilled in the art such as, for example, a physician, a veterinarian, a scientist, and other medical and research professionals. For example, one skilled in the art can begin with a low dosage that can be increased until reaching the desired treatment outcome or result. Alternatively, one skilled in the art can begin with a high dosage that can be decreased until reaching a minimum dosage needed to achieve the desired treatment outcome or result.

In suitable embodiments, the subject is administered an APE1/Ref-1 inhibitor in amounts ranging from about 1.0 μM to about 125 μM inhibitor, including from about 1.0 μM to about 50 μM inhibitor. In one particular embodiment, the inhibitor is APX3330, and the subject is administered an amount of from about 1.0 μM to about 50 μM APX3330.

In some embodiments, the APE1 inhibitor is administered via a composition that includes the APE1 inhibitor and a pharmaceutically acceptable carrier. Pharmaceutically acceptable carriers may be, for example, excipients, vehicles, diluents, and combinations thereof. For example, where the compositions are to be administered orally, they may be formulated as tablets, capsules, granules, powders, or syrups; or for parenteral administration, they may be formulated as injections (intramuscular, subcutaneous, intramedullary, intrathecal, intraventricular, intravenous, intravitreal), drop infusion preparations, or suppositories. These compositions can be prepared by conventional means, and, if desired, the active compound (e.g., APX3330) may be mixed with any conventional additive, such as an excipient, a binder, a disintegrating agent, a lubricant, a corrigent, a solubilizing agent, a suspension aid, an emulsifying agent, a coating agent, or combinations thereof.

It should be understood that the pharmaceutical compositions of the present disclosure can further include additional known therapeutic agents, drugs, modifications of the synthetic compounds into prodrugs, and the like for alleviating, mediating, preventing, and treating the diseases, disorders, and conditions described herein. For example, in one embodiment, the APE1 inhibitor can be administered with one or more of current therapeutic agents and drugs for treating IBD (e.g., 5-aminosalicylic acid (5-ASA), corticosteroids, azathioprine, 6-mercaptopurine, methotrexate, cyclosporine, tacrolimus, anti-TNF drugs (e.g., infliximab, certolizumab, adalimumab, and golimumab), vedolizumab, natalizumab, ustekinumab, probiotics, antibiotics, and anti-inflammatories (e.g., mesalamine (Asacol HD, Delzicol, others), balsalazide (Colazal) and olsalazine (Dipentum), and the like).

The pharmaceutical compositions including the APE1 inhibitor and/or pharmaceutical carriers used in the methods of the present disclosure can be administered to a subset of individuals/subjects in need. As used herein, a “subject in need” refers to an individual at risk for or having inflammatory and/or chronic pain of the gut, or an individual at risk for or having a disease or disorder associated with inflammation and/or chronic pain (e.g., functional gastroinstestinal disease, indeterminate colitis (IC), inflammatory bowel disease (IBD) (e.g., ulcerative colitis (UC, Crohn's disease (CD))). Additionally, a “subject in need” is also used herein to refer to an individual at risk for or diagnosed by a medical professional as having inflammatory or chronic pain. As such, in some embodiments, the methods disclosed herein are directed to a subset of the general population such that, in these embodiments, not all of the general population may benefit from the methods. Based on the foregoing, because some of the method embodiments of the present disclosure are directed to specific subsets or subclasses of identified individuals (that is, the subset or subclass of subjects “in need” of assistance in addressing one or more specific conditions noted herein), not all individuals will fall within the subset or subclass of individuals as described herein. In particular, the individual in need is a human. The individual in need can also be, for example, a research animal such as, for example, a non-human primate, a mouse, a rat, a rabbit, a cow, a pig, and other types of research animals as known in the art, or a domestic animal such as, for example, dog, cat, and other domestic animal known to those skilled in the art.

Various functions and advantages of these and other embodiments of the present disclosure will be more fully understood from the examples shown below. The examples are intended to illustrate the benefits of the present disclosure, but do not exemplify the full scope of the disclosure.

### Example

In this Example, the murine model of IBD named Winnie, in which spontaneous chronic colitis results from a primary intestinal epithelial defect conferred by a mutation in the Muc2 mucin gene, was used to analyze the symptoms of IBD and the effects of treatment with APX3330.

Materials and Methods

Although other models of chronic intestinal inflammation have been developed (e.g., IL-10 knockout), most of them are environment-dependent (attain inflammation only in the presence of pathogenic bacteria). Further, unlike other models, mild spontaneous inflammation in the colorectum is developed in all Winnie mice by 6 weeks of age (young adults) in pathogen-free conditions; it progresses over time and results in severe colitis by the age of 12-16 weeks. This is due to a thinner mucus layer allowing increased intestinal permeability and thus enhanced susceptibility to luminal toxins normally within the gut. In humans, Muc2 production and secretion are reduced leading to a thinner mucosal layer and increased intestinal permeability. Winnie mice (Win/Win) display symptoms of diarrhoea (not watery), ulcerations, rectal bleeding and pain at the acute stages of colitis similar to those in human IBD.

APX3330 (also referred to herein as “E3330”) was synthesized per previous publications (e.g., J Med Chem. 2010 Feb. 11; 53(3): 1200-1210), dissolved in N,N-dimethylformamide (Sigma-Aldrich) and stored as a 40 mM stock at −80° C. Lipopolysaccharides (LPS) from Escherichia coli 0111:B4 was purchased from Sigma-Aldrich Inc. (St. Louis, Mo.), dissolved in MPL and stored as a 50 mM at −20° C. for a month. Recombinant rat CCL2/MCP-1 protein was purchased from R&D Systems (Minneapolis, Minn.), dissolved in PBS and stored at −20° C. for up to a month. The TLR4 antagonist, LPS-RS, was purchased from Invivogen, dissolved in MPL and stored at −80° C. The CCR2 antagonist, RS 504393, was purchased from Sigma-Aldrich Inc. (St. Louis, Mo.), dissolved in MPL and stored at −20° C. for a month. Before drug treatment, the stocks were diluted in F-12 growth medium and added to cultures and incubated for 2-96 hours as indicated.

Animals

The Winnie murine model of chronic colitis (12 w.o.; 15-35 g; n=24) were attained from Victoria University Werribee Animal Facility (Melbourne, Australia) to determine the effectiveness of APX3330 on mechanisms of oxidative stress. Treated Winnie mice were paralleled to a control C57BL/6 group (12 w.o.; 20-30 g; n=12) inclusive of female and male mice. All mice were acclimatized for 3 days prior to receiving in vivo intraperitoneal (IP) injections. All mice were housed at Western Centre for Health, Research and Education (WCHRE, Melbourne, Victoria, Australia) in a temperature-controlled environment with a 12-hour day/night cycle. All animals had free access to food and water with minimal efforts made to minimize any suffering. All experimental procedures in this Example were conducted in agreement to the Australian National Health and Medical Research Council (NHMRC) guidelines and approved by Victorian University Animal Experimentation Ethic Committee (AEEC) under animal ethics AEETH 13/001 and AEC 17/016.

Administration of APX3330

The small molecular APE1/Ref-1 antagonist; APX3330, was administered via IP injections in Winnie mice at a dose of 25 mg/kg (30 G needle, max volume 200 μl) dissolved in Cremphore (2%) (Sigma-Aldrich): Ethanol (2%) in sterile water (96%). Mice received alternating IP injections twice a day with 12-hour intervals, over the course of two weeks during predominate intestinal inflammation. Winnie-sham treated mice received vehicle injections excluding APX3330 drugs. All mice were monitored, weighed and faecal pellets were collected over the course of treatment.

Gastrointestinal Transit

GI transit was acquired via a non-invasive radiological method. Briefly, control C57BL/6, Winnie-sham treated and Winnie-APX3330 treated mice received an administration of barium sulfate (2.5 mg/mL; max volume of 200 μl; X-OPAQUE-HD) via oral gavage. Sequential x-rays were attained by HiRay Plus Porta610HF x-ray apparatus (JOC Corp, Kanagawa, Japan; 50 kV, 0.3 mAs, exposure time 60 ms) immediately post Barium sulfate administration (0 min), every 5 minutes for the first hour, 10 minutes for the second hour and then every 20 minutes through to 250 minutes. Fujifilm FCR Capsule XL11 and analysed on eFilm 4.2.0 software developed images. Parameters of GI transit were measured by time (mins) to determine contrast passing through whole GI tract (whole transit time), stomach to caecum (oro-cecal transit time; OCTT), leaving caecum to anus (colonic transit time; CTT) and caecum retention time.

Assessment of Intestinal Permeability

On day 14, mice were subjected to an overdose of lethobarb (1:16 dilution, 30 G, 100 μl/20 g) IP injections for blood collections prior to harvesting colon tissues. Puncture allowed for a minimal collection of 600 μl of blood via a 26 G needle. Blood was kept on ice for 2 hours, centrifuged at 12×G at 4° C. for 15 minutes were plasma was collected and stored at −20° C. for subsequent ELISA experiments. Quantikine ELISA (mouse/rat FABP1/L-FABP) (Abcam) measured sera levels for fatty acid binding protein (FABP)-1. All samples were repeated in duplicates for statistical value. Assay diluent RD1-17 (50 μL) was added to each well, followed by 50 μL of standard, 10 μl acquired from either control C57BL/6, Winnie-sham treated and Winnie-APX3330 treated mice blood sera. Plate was briefly mixed, incubated at room temperature for 2 hours on a horizontal orbital microplates shaker set at 200±50RPM. Each well was then aspirated and washed with 400 μL of wash buffer prior to adding 100 μL of mouse/rat FABP1 conjugate. Samples were incubated as above. Each well was then aspirated and immersed with 100 μL of substrate solution and incubated for 30 minutes at room temperature protected from light followed with 100 μL of a stop solution. Microplate reader capable of measuring absorbance at 450 nm, with the correction wavelength set at 540 nm was used to detect FABP-1 protein (ng/mL) in blood sera.

Assessment of Intestinal Inflammation

Faecal lipocalin (Lcn)-2 ELISA kit (Abcam) were used to detect efficacy of APX3330 on levels of colonic inflammation. Faecal samples collected on day 14 of treatments from control C57BL/6, Winnie-Sham treated and Winnie-APX3330 treated mice were reconstituted in PBS-0.1 TWEEN 20 (100 mg/mL) to form a homogenous faecal suspension. Homogenous suspension was centrifuged for 10 minutes at 12000 RPM at 4° C. Lcn-2 were determined in the clear supernatants. All samples were repeated in duplicates for statistical value. Assay diluent 5B (50 μL) was added to each well, followed by 50 μL of standard, 10 μl acquired from either control C57BL/6, Winnie-sham treated and Winnie-APX3330 treated mice. Plate was briefly mixed, incubated at room temperature for 1 hour on a horizontal orbital microplates shaker set at 400±50 RPM. Each well was then aspirated and washed with 350 μL of wash buffer prior to adding 100 μL of TMB substrate. Samples were incubated as above. Each well was then aspirated and immersed with 100 μL of substrate solution and incubated for 10 minutes at room temperature protected from light followed with 100 μL of a stop solution. Microplate reader capable of measuring absorbance at 450 nm, with the correction wavelength set at 540 nm was used to detect Lcn-2 protein (pg/mL) in faecal pellet supernatant.

Whole Organ Bath Experiments of Isolated Colonic Motility

Colonic motility experiments were completed ex vivo. Whole colons were removed from control C57BL/6, Winnie-sham treated and Winnie-APX3330 treated mice. Colons were positioned horizontally cannulated at the oral and anal end in an organ bath superfused with carbogenated (composition in mM: NaCl 118, KCl 4.6, CaCl₂ 3.5, MgSO₄ 1.2, NAH₂PO₄ 1, NaHCO₃ 25 and d-Glucose; carbongenated with 95% O₂ and 5% CO₂) ×1 Krebs solution whilst maintained at a temperature of 37° C. The oral cannula was connected to a reservoir with 1× Krebs solution that was adjusted to maintain intraluminal pressure (0 to +2 cm H₂O). The anal end was coupled to an out-flow tube with a maximum 2 cm H₂O backpressure. A recording was made by a video camera being positioned above the organ bath, this recorded the colonic contractile activity. Tissue were left to equilibrate for 30 minutes prior to 2× 20-minute recordings at increasing intraluminal pressure. Videos were transposed into spatiotemporal maps with Scribble v2.0 software and were analysed by using MATLAB, v2017a software to assess parameters of colonic motility.

Tissue Collections

Distal colon tissues were harvested in oxygenated physiological saline, flushed of faecal content and cut along the mesenteric border. Tissues were pinned down with mucosal side upwards in a Slygard-lined petri dish and were briefly fixed with Zamboni's fixative (2% formaldehyde containing 0.2% picric acid) over night at 4° C. Zamboni's fixative was removed by a serial of washes (3×10 minutes) with dimethyl sulfoxide (DMSO) (Sigma-Aldrich, Sydney, Australia) followed by (3×10 minutes) with 1× phosphate buffered solution (PBS). Tissues were processed for cross sections wholemount longitudinal muscle-myenteric preparations (LMMP). For histological staining, β-tubulin III and α-Smooth muscle actin (SMA) labelling distal colon tissues were pinned and fixed as above without stretching and stored in 50:50 optimum cutting temperature (OCT) compound (Tissue Tek, CA, USA) and frozen in liquid nitrogen-cooled isopentane and OCT and stored at −80° C. until cryo-sectioned (20 μm) onto glass slides for immunohistochemistry (IHC). For immunolabeling glial fibrillary acidic protein (GFAP), microtubule associated protein-2 (MAP2), HMGB1 and APE1, the distal colon LMMPs were stretched to maximum capacity without tearing in Slygard-lined petri dishes and were subjected to fixation and washes as above. To expose the myenteric plexus, removal of mucosa, submucosa and circular muscle was performed prior to IHC.

Immunohistochemistry and Histology

Immunohistochemistry (IHC) was completed. Specimens were subjected to a one hour incubation at room temperature with 10% normalised donkey serum (NDS) (Merck Millipore, Australia) prior to labelling with primary antibodies (Table 2) in distal colon cross sections and LMMPs. Sections and preparation were washed with 1×PBS (3×10 minutes) and then briefly incubated with fluorophore-conjugated secondary antibodies (Table 2). All specimens were stained with 4′,6′-diamindino-2-pheylindole dihydrochloride (DAPI) to identify immunoreactive cells. Tissues were mounted on glass slides with fluorescent mounting medium (DAKO, North, Sydney, NSW, Australia Tissues for histology were cryo-sectioned at 10 μm, cleared and rehydrated in graded ethanol concentration. For standard hematoxylin and eosin stain (H&E) and Alcian blue stain, sections were immersed in histolene (3×4 minutes), 100% ethanol (2 minutes), 95% ethanol (2 minutes), 70% ethanol (2 minutes), rinsed in tap water (30 seconds), then in hematoxylin (Sigma-Aldrich) (1 minute) or Alcian blue (Sigma-Aldrich) (30 minutes), rinsed in tap water, immersed in Scott's tap water (1 minute) and eosin (Sigma-Aldrich) (5 minutes), rinsed in tap water, immersed in 100% ethanol (2×1 minute) and histolene (4 minutes) and finally mounted on glass slides with DPX mountant. A histological grading system evaluated gross morphological damage on constraints inclusive of mucosal flattening (0=normal, 3=severe flattening), manifestations of haemorrhagic sites (0=none, 3=frequent), damage or distortion to circular muscle (0=normal, 3=substantial thickening and disorganization of muscle layer) and loss to goblet cells (0=none, 3=depleted cells). All slides were coded, and analysis was performed blindly.

TABLE 2
Antibodies used in this Exampoe for Immunohistochemistry Techniques

| Primary | | Host | | | | Secondary | Host | | |
|---|---|---|---|---|---|---|---|---|---|
| Antibody | Target | Specie | Titration | Specimen | Supplier | Antibody | Specie | Titration | Supplier |
| Anti-α- | Smooth | Rabbit | 1:1000 | Cross- | Abcam | Alexa Fluor | Donkey | 1:200 | JacksonImmunoResearch |
| SMA | muscle cells | | | sections | | 647 | anti-rabbit | | |
| Anti-β- | Nerve fibers | Rabbit | 1:1000 | Cross- | Abcam | Alexa Fluor | Donkey | 1:200 | JacksonImmunoResearch |
| tubulin III | | | | sections | | 488 | anti-rabbit | | |
| Anti- | Neurons | Chicken | 1:5000 | LMMP | Abcam | Alexa Fluor | Donkey | 1:200 | JacksonImmunoResearch |
| MAP2 | | | | | | 488 | anti-rabbit | | |
| Anti- | Glial Cells | Goat | 1:500 | LMMP | Abcam | Alexa Fluor | Donkey | 1:200 | JacksonImmunoResearch |
| GFAP | | | | | | 594 | anti-goat | | |
| Anti- | APE1 protein | Mouse | 1:1000 | LMMP | Provided by | Alexa Fluor | Donkey | 1:200 | JacksonImmunoResearch |
| APE1 | | | | | Professor | 648 | anti-mouse | | |
| | | | | | Mark Kelley | | | | |
| Anti- | Pro-inflammatory | Mouse | 1:500 | LMMP | Abcam | Alexa Fluor | Donkey | 1:200 | JacksonImmunoResearch |
| HMGB1 | response | | | | | 648 | anti-mouse | | |
| Anti-8- | DNA Damage | Mouse | 1:500 | LMMP | Abcam | Alexa Fluor | Donkey | 1:200 | JacksonImmunoResearch |
| OHdG | | | | | | 648 | anti-mouse | | |

Imaging and Quantitative Analysis

Confocal microscopy (Nikon Eclipse Ti multichannel confocal laser scanning system, Nikon, Japan) acquired immunolabelled sections. Triple-labelled specimens were visualized and imaged by using filter combinations of FITC, Alexa 594 and Alexa 647 (488-nm, 559-nm or 640.4-nm excitation respectively). Images (512×512 pixels) were obtained at 20× (dry, 0.75) or 40× (oil immersion, 1.3) lenses. Percentage for nerve fiber density and IR-α-SMA were quantified in colon cross sections (total area 1 mm² area, 4× images taken randomly at ×20 magnification). All images were acquired at an equal acquisition, exposure-time conditions, calibrated to a standard minimum baseline fluorescence and converted to binary. In colon cross sections and LMMPs alterations nerve fibers and IR-GFAP glial cells respectively, were evaluated by analysing changes to fluorescence, converted to binary and changes in fluorescence were measured as a percentage (%) using ImageJ software (National Institute of Health, Bethesda, Md., USA). Total number of myenteric neurons IR for MAP2, APE1-IR and HMGB1-IR cells were colabelled with DAPI and quantified within 10 ganglia per each preparation with ImageJ software (National Institute of Health) at ×40 magnification. Damage to colonic morphological was evaluated in H&E and Alican blue stained sections with a Zeiss Axio Imager Microscope and Images were capture with MetaSystems Metafer program. A VSlider software stitched images together.

Superoxide Production in the Myenteric Plexus

MITOSOX™ Red M36008 (Invitrogen, Australia), acquired mitochondrial-derived production of superoxide in the myenteric ganglia. Briefly, distal colon preparations from control C57BL/6, Winnie-sham treated and Winnie-APX3330 treated mice freshly excised to expose the myenteric plexus. Samples were incubated at 37° C. for 40 minutes with 5 μM MITOSOX™ Red M36008. Tissues were washed with oxygenated physiological saline and fixed with Zamboni's fixative for 1-hour followed by (3×10 minute) 1×PBS washes. Prepared tissues were mounted on glass slides with DAKO fluorescent a mounting medium for imaging Images were captured as previously described above. Images were converted into binary and changes in fluorescence were measured in arbitrary units (arb. Units) relative to ganglion area using ImageJ software (National Institute of Health).

RNA Isolation and NGS Arrays

Control C57BL/6, Winnie-sham treated and Winnie-APX3330 fresh colon tissues were collected and snap frozen into liquid nitrogen. Samples were then sent to the Australian Genome Research Facility (AGRF) where next generation sequencing (NGS) was completed. NGS will be carried out by removing 2-3 μg of RNA per sample (≥100 ng/μl). The integrity of the RNA will be assessed by a Bioanalyzer as samples are to have a RNA integrity number (RIN) value of ≥8.0. The RNA samples went through a DNase treatment ready for library preparation. Bioinformatics will be run to determine the biological significance and filtration through RNA sequencing for expression analysis. Samples underwent quality and adapter trimming, alignment, quantification and normalization. Results gathered from bioinformatics consisted of sequences, alignments, post alignments, transcripts assemblies and gene count files. Final data obtained from AGRF were analysed and interpreted to determine APE1/Ref-1 associated pathways.

### Results

APX3330 Improved Clinical Symptoms in Winnie Mice

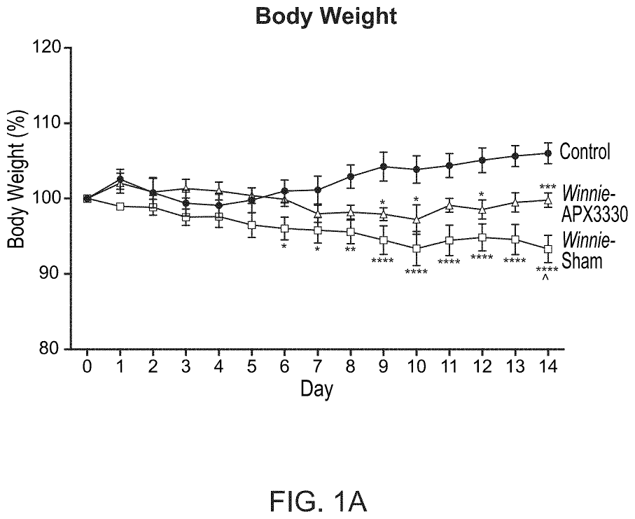

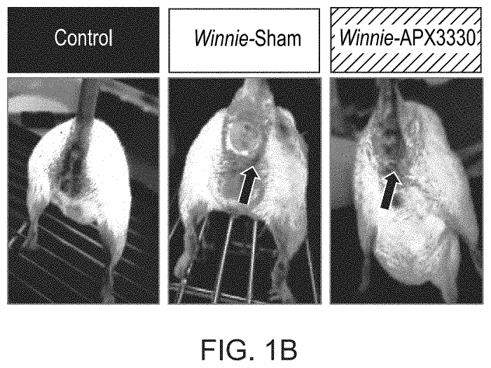

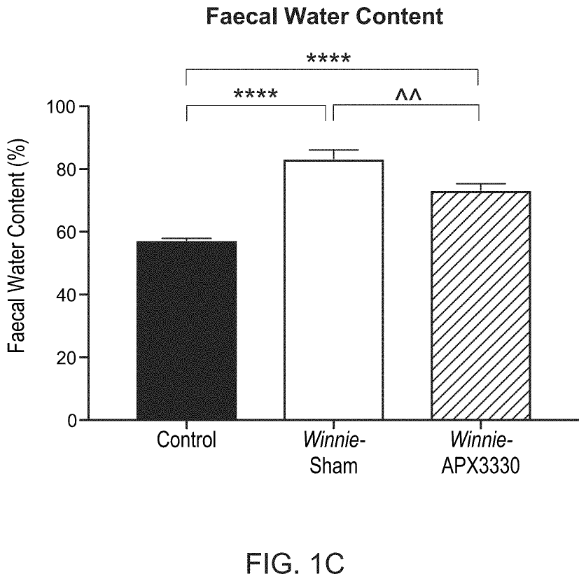

Changes in animal body weights, severity of intestinal inflammation indicated by presence of rectal prolapse, and faecal water content were measured to assess the effects of the treatment. Clinical symptoms were observed in experiential groups on day 14 (FIGS. 1A-1C). Body weights were attained over the 14 day period from control C57BL/6, Winnie-sham treated and Winnie-APX3330 treated mice (Table 3, FIG. 1A). Specifically, Winnie-sham treated mice showed progressively decreasing body weight from day 6 until day 14 compare to control C57BL/6 mice (Table 3, FIG. 1A). On day 14 Winnie-APX3330 treated mice body weight improved when compared to Winnie-sham treated mice (P<0.05) though not comparable to control C57BL/6 mice (Table 3, FIG. 1A). Prolapse observed rectal protrusion evident in Winnie-sham treated animals had rectal prolapse evident as a protrusion with edema and bleeding (FIG. 1B). Conversely, Winnie-APX3330 treated mice demonstrated reduced rectal prolapse, edema and bleeding by day 14 of treatments (FIG. 1B). Fresh faecal pellets were collected on day 14 to assess water content (FIG. 1C). Winnie-sham treated mice had a high level faecal water content (83.4±2.7%, P<0.0001, n=7) compared to control C57BL/6 mice (57.3±0.6%, n=7) (FIG. 1C). Conversely, faeces from Winnie-APX3330 treated mice (73.2±2.1%, P<0.01, n=7) had a lower faecal water retention when compared to Winnie-sham treated animals (FIG. 1C). However, faecal pellets obtained from Winnie-APX3330 treated animals demonstrated higher water retention compared to control C57BL/6 mice (P<0.0001) (FIG. 1C).

TABLE 3
Daily Body Weight Assessment over the 14-Day Period

| Day | 1 | 2 | 3 | 4 | 5 | 6 | 7 | 8 | 9 | 10 | 11 | 12 | 13 | 14 |
|---|---|---|---|---|---|---|---|---|---|---|---|---|---|---|
| Control | 102 ± | 100 ± | 99 ± | 99 ± | 99 ± | 100 ± | 101 ± | 102 ± | 104 ± | 103 ± | 104 ± | 105 ± | 105 ± | 106 ± |
| (n = 10) | 1.3 | 1.9 | 1.7 | 1.6 | 1.6 | 1.5 | 1.9 | 1.5 | 1.9 | 1.8 | 1.6 | 1.6 | 1.4 | 1.4 |
| Winnie- | 99 ± | 99 ± | 98 ± | 98 ± | 98 ± | 96 ± | 96 ± | 96 ± | 94 ± | 93 ± | 94 ± | 95 ± | 95 ± | 93 ± |
| sham | 0.4 | 1.0 | 1.1 | 1.5 | 1.5 | 1.6 | 1.5 | 1.7 | 1.9 | 2.3 | 2.0 | 1.8 | 2.0 | 1.8 |
| (n = 12) | | | | | | * | * | ** | **** | **** | **** | **** | **** | ****^{∧} |
| Winnie- | 102 ± | 100 ± | 101 ± | 101 ± | 100 ± | 100 ± | 98 ± | 98 ± | 98 ± | 99 ± | 99 ± | 99 ± | 99 ± | 100 ± |
| APX3330 | 1.3 | 1.9 | 1.3 | 1.3 | 1.0 | 0.9 | 1.1 | 0.9 | 0.9 | 0.9 | 1.3 | 1.3 | 1.3 | 0.9 |
| (n = 8) | | | | | | | | | * | * | | * | * | * |

Data expressed as mean +/− SEM, ***P <0.0001, **P <0.001, *P <0.05, when compared to controls: ^{∧}P <0.05 when compared to Winnie-APX3330 treated

APX3330 Improved GI Functions in Winnie Mice

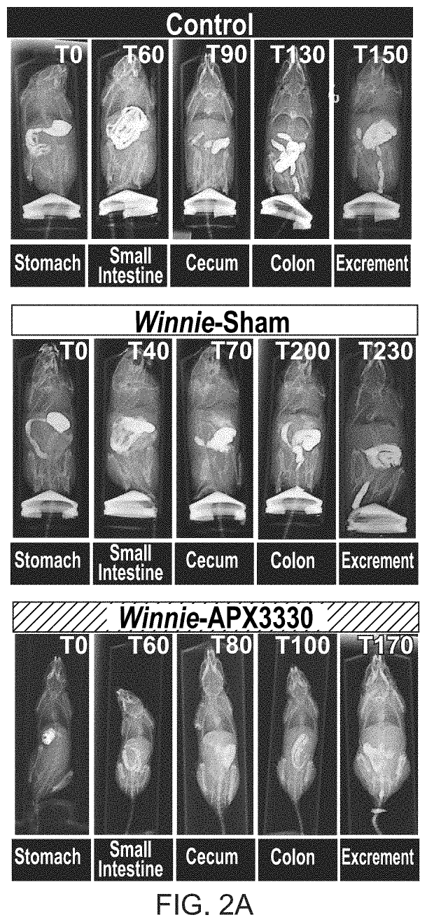

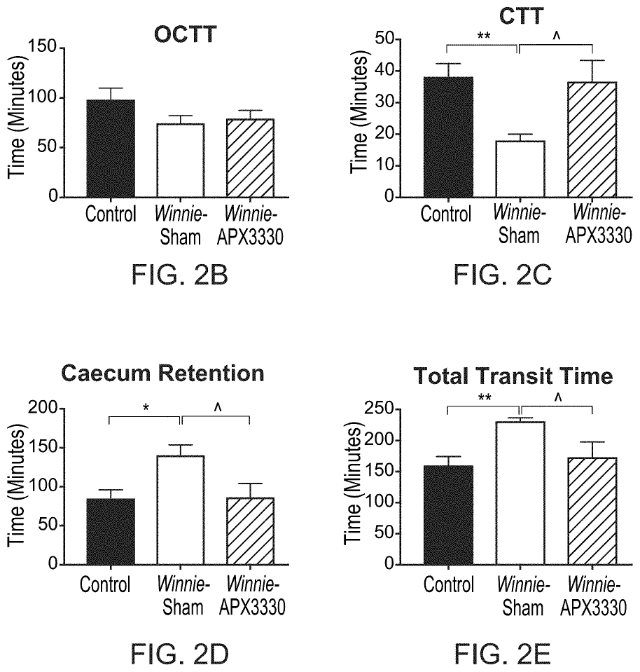

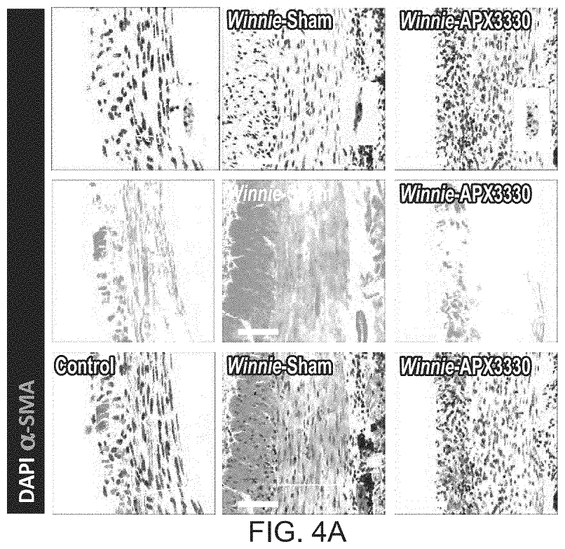

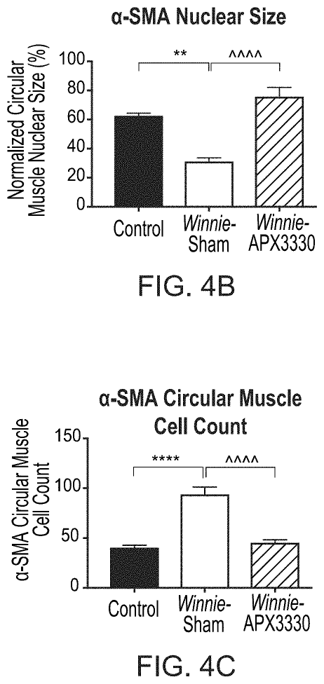

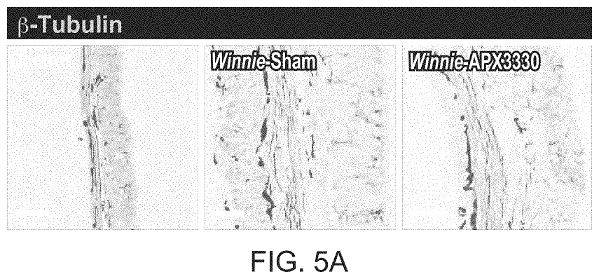

The efficacy of APX3330 was assessed on parameters of GI transit and colonic motility. Radiographic images captured barium sulfate from the stomach to first pellet in control C57BL/6, Winnie-sham treated and Winnie-APX3330 treated mice (FIG. 2A). No changes were observed in OCTT time between experimental groups (FIG. 2B); however, other parameters of GI transit were found compromised in Winnie-sham treated mice and restored in Winnie-APX3330 treated mice. Winnie-sham treated mice demonstrated an accelerated CTT time (18.0±2.0 mins, P<0.01, n=10) compared to control C57BL/6 mice (38.0±4.2 mins, n=10). However, Winnie-APX3330 treated mice displayed an improved CTT time compared to Winnie-sham treated mice (FIG. 4C). It was evident that Winnie-sham treated mice (36.7±6.7 mins, P<0.05, n=7) had a prolonged retention time in the cecum (140.5±13.2 mins, P<0.05) compared to control C57BL/6 (85.0±11.0 mins) and Winnie-APX3330 treated mice (86.4±17.8 mins, P<0.05) (FIG. 4D). APX3330 treatment reduced the total transit time (173.3±24.6 mins, P<0.05) in Winnie mice to the level of C57BL/6 mice (FIG. 4E).

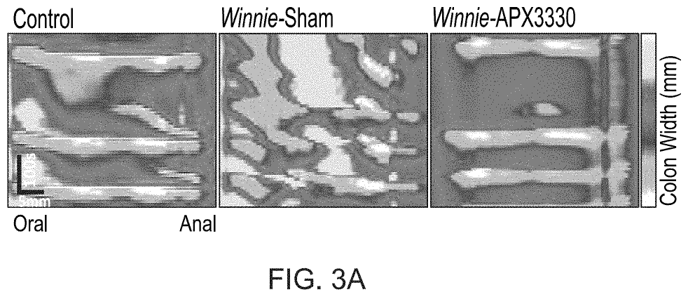

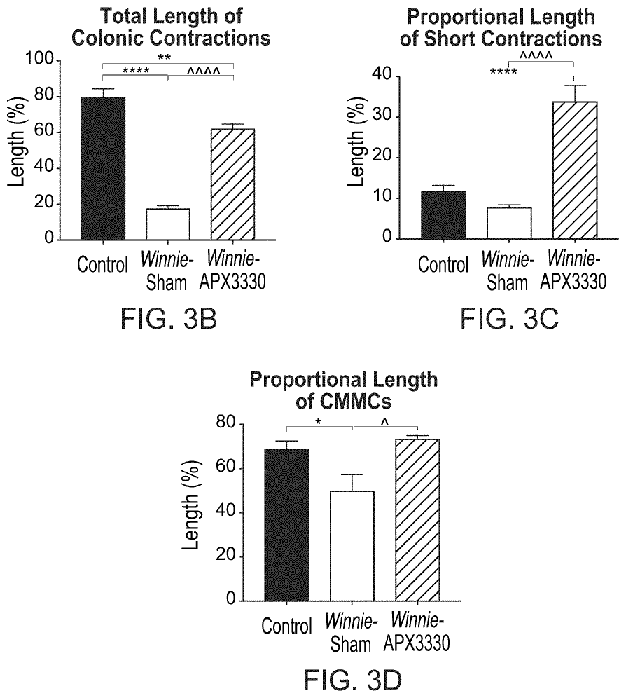

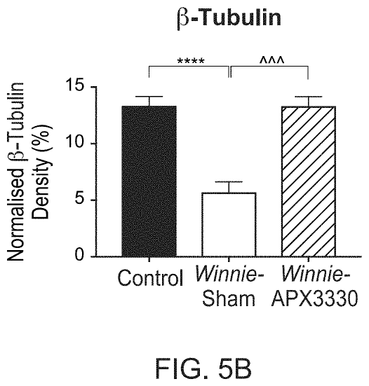

Ex Vivo whole organ bath experiments assessed the effects of APX3330 on colonic contractile activity (FIGS. 3A-3D). Colonic migrating motor complexes (CMMCs) were identified as a contraction>50% initiated from oral to anal end of the colon length. However, short contractions were defined as contractions that are <50% of the colon length. Video recordings were transferred into spatiotemporal maps with contraction considered as a line (FIG. 3A). Overall, the total length of colonic contractions in Winnie-sham mice (17.9±1.4%, P<0.0001, n=5) were significantly decreased compared to control C57BL/6 mice (79.9±4.5%, n=5) and in Winnie-APX3330 mice (62.3±2.4%, P<0.0001, n=5), although not to the level in the colons from control C57BL/6 mice (P<0.01) (FIG. 3B). No changes to short contraction lengths were observed between control C57BL/6 mice (11.8±1.4%, n=7) and Winnie-sham treated mice (7.9±0.5%, n=7) (FIG. 5C). However, Winnie-APX3330 treated mice (33.9±3.8%, n=5) had significantly increased proportional short contraction length compared to control C57BL/6 mice (P<0.0001) and Winnie-sham treated animals (P<0.0001) (FIG. 3C). Length of CMMCs in Winnie-sham treated mice (50±7.0%, P<0.05, n=5) were significantly attenuated compared to control C57BL/6 mice (68±3%, n=5) (FIG. 3D). Treatment with APX3330 restored proportional lengths of CMMCs (73±1.0%, P<0.01, n=5) compared to Winnie-sham treated mice to the level of control C57BL/6 mice (FIG. 3D).

APX3330 subsided changes to smooth muscle cell morphology and number in Winnie Mice

Changes seen to smooth muscle cells may contribute to the functional changes foreseen in the inflamed colon. An anti-α-SMA antibody co-labelled with DAPI identified smooth muscle cells in distal colon cross sections obtained from control C57BL/6, Winnie-sham treated and Winnie-APX3330 treated animals (FIGS. 4A-4C). Winnie-sham treated animals (31.1±2.6%, P<0.01, n=5) showed decreased size of α-SMA-immunoreactive (IR) to nuclear size compared to control C57BL/6 mice (62.6±1.7%, n=5) (FIG. 4B). However, in Winnie-APX3330 treated mice smooth muscle cell was improved (75.7±6.4%, P<0.0001, n=5) compared to Winnie-sham treated animals with sizes similar to control C57BL/6 mice (FIG. 4B). The number of α-SMA-IR co-labelled DAPI smooth muscle cells quantified within the circular muscle was increased in Winnie-sham treated mice (93.8±7.3, P<0.0001, n=4) when compared to control C57BL/6 mice (40.4±2.5, n=5) (FIG. 4C). Conversely, Winnie-APX3330 treated mice had a significant decreased number of α-SMA-IR cells (45.2±3.1, P<0.05, n=5) compared to Winnie-sham treated mice but similar to control C57BL/6 mice in the circular muscle of the distal colon (FIG. 2C).

APX3330 Treatment Restored Nerve Fiber Density in the Winnie Mice

A β-tubulin (III) antibody specific for neuronal microtubule protein stained nerve fibres innervating in cross sections of the distal colon from control C57BL/6, Winnie-sham treated and Winnie-APX3330 treated animals (FIGS. 5A & 5B). Winnie-sham treated mice demonstrated a significant decrease to nerve fibre density (5.7±0.9%, P<0.0001, n=9) compared to control C57BL/6 mice (13.4±0.8%, n=8) in the distal colon (FIGS. 5A & 5B). However, Winnie-APX3330 treated mice had resorted nerve fibre density in the colon (13.3±0.8, P<0.001, n=5) as compared to control C57BL/6 mice levels (FIGS. 5A & 5B).

APX3330 Treatment Improved Gross Morphology and Goblet Cell Density in the Colon of Winnie Mice

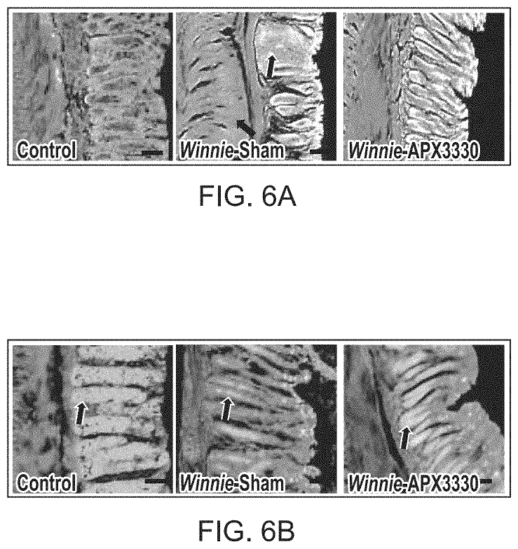

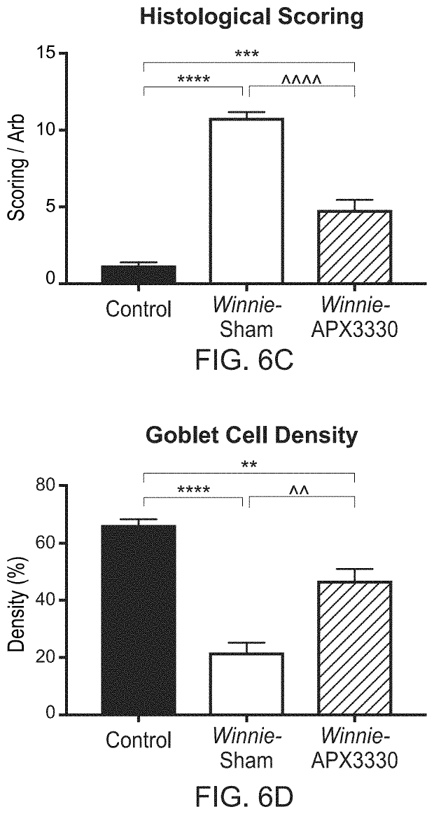

Gross morphological changes to the colon were assessed via a H&E stain in the inflamed colon (FIG. 6A). Normal organization of the epithelium with elongated crypts was observed in control C57BL/6 mice. Conversely, Winnie-sham treated animals demonstrated mucosal flattening, leukocyte manifestation and hyperplasia to the smooth muscle wall. APX3330 treatment in Winnie mice appeared to attenuate damage to the colon foreseen with restored architecture. In addition, an Alcian blue stain was performed to determine loss to goblet cells in control C57BL/6, Winne-sham treated and Winnie-APX3330 treated animals (FIG. 6B). It was evident that Winnie-sham treated mice displayed a higher histological grading (10.8±0.4, P<0.0001, n=5) compared to control C57BL/6 mice (1.2±0.2, n=5) (FIGS. 6A & 6C). However, Winnie-APX3330 treated mice displayed an improved histological grading (4.8±0.7, n=5) when compared to Winnie-sham mice (P<0.0001) although not comparative to control C57BL/6 mice (P<0.001) (FIGS. 6A & 6C). A loss to goblet cell density was quantified in Winnie-Sham treated mice (22.0±3.3%, P<0.0001, n=5) compared to control C57BL/6 mice (65.7±2.7%, n=5) (FIGS. 6B & 6D). However, Winnie-APX3330 treated mice displayed an improved goblet density (46.5±4.3%, n=4) compared to Winnie-sham treated mice (P<0.01), although not comparable to control C57BL/6 mice (P<0.01) (FIGS. 6B & 6D).

APX3330 Treatment Alleviated Intestinal Permeability and Inflammation in Winnie Mice

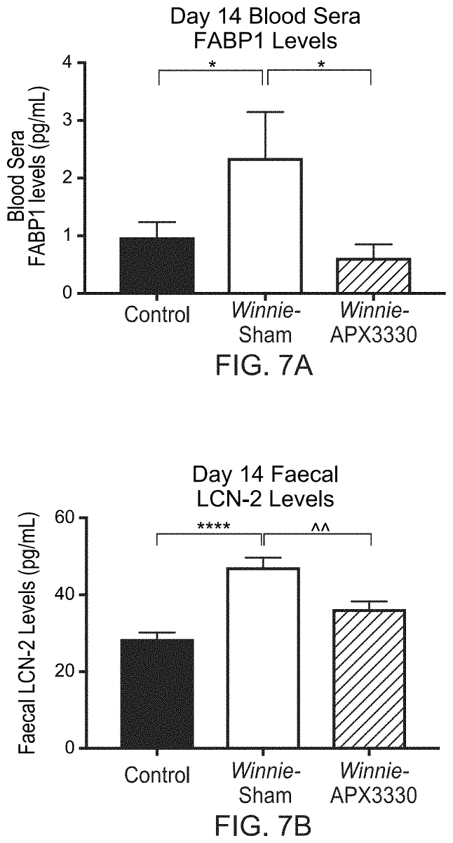

FABP1 has a major role in phospholipid synthesis and supporting epithelial barrier integrity in the intestines. Increased levels of FABP1 in the blood sera result from increased intestinal permeability. In Winnie-sham treated mice FABP1 in blood sera was higher (2.3±0.7 ng/mL, P<0.05, n=4) compared to control mice (0.6±0.1 ng/mL, n=5) (FIG. 7A). However, levels for FABP1 in Winnie-APX3330 mice had decreased in blood sera (0.6±0.2 ng/mL, P<0.05, n=5) compared to Winnie-sham treated animals (FIG. 7A). Lcn-2 is a non-invasive biomarker for intestinal inflammation. At day 14, Winnie-sham mice displayed an increase in Lcn-2 faecal level (47±2.5 pg/mL, P<0.0001, n=8) compared to control C57BL/6 mice (28±1.7 pg/mL, n=7) (FIG. 7B). Treatment with APX3330 decreased Lcn-2 faecal levels (36±2.0 pg/mL, P<0.01, n=5) compared to Winnie-sham treated mice (FIG. 7B).

APX3330 Treatment Improved Glial Cell Density in the Myenteric Plexus

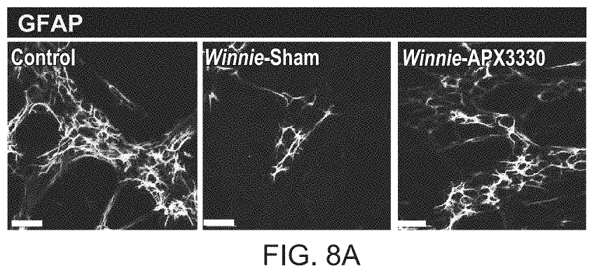

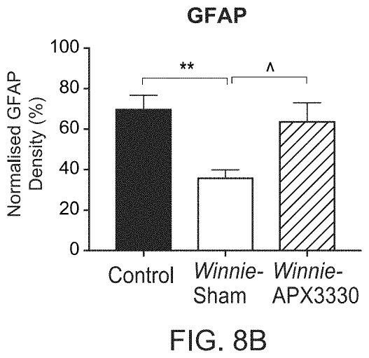

The density of Glial cell IR for GFAP was assessed in the myenteric plexus of the distal colon (FIGS. 8A & 8B). Glial cell density was represented relative to the ganglion area in LMMP preparations from control C57BL/6, Winnie-sham treated and Winnie-APX3330 treated mice (FIG. 8A). Winnie-sham treated mice had a significant decrease in glial cell density (36.3±3.6%, P<0.01, n=5) compared to control C57BL/6 mice (70.2±6.5%, n=5) (FIGS. 8A & 8B). APX3330 treatment of Winnie mice had restored GFAP density (64.2±8.8%, P<0.05, n=4) when compared to Winnie-sham treated mice (FIGS. 8A & 8B).

APX3330 Treatment Alleviated Myenteric Neuronal Loss in the Distal Colon

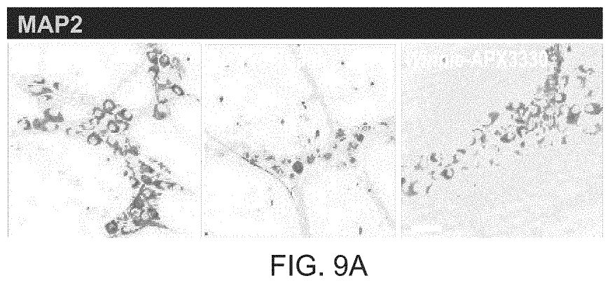

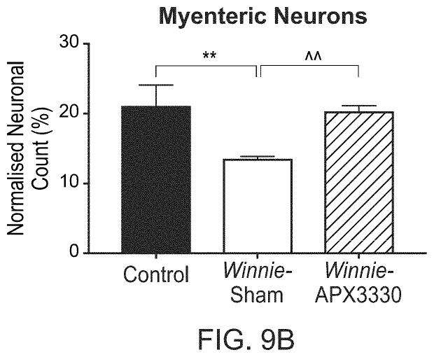

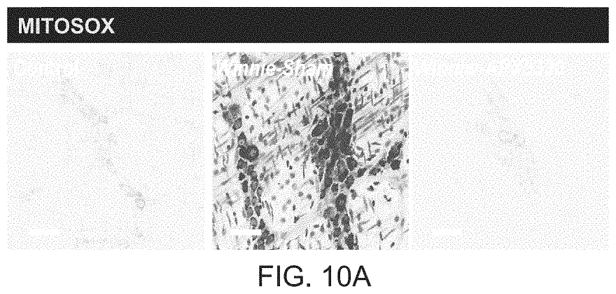

Myenteric neurons were identified with an anti-MAP2 pan neuronal marker in LMMP preparations from control C57BL/6, Winnie-sham and Winnie-APX3330 mice (FIG. 9A). In Winnie-sham treated mice (22.9±2.4, P<0.01, n=6) there was a significant decrease in proportional number of myenteric neurons compared to control C57BL/6 mice (37.6±3.3, n=4) (FIGS. 9A & 9B). Winnie-APX3330 treated mice (31.9±2.2%, P<0.05, n=7) had alleviated complete loss to myenteric neurons compared to Winnie-sham mice (FIGS. 9A & 9B).

APX3330 Treatment Reduced Superoxide Production in the Inflamed Myenteric Ganglia of Winnie Mice

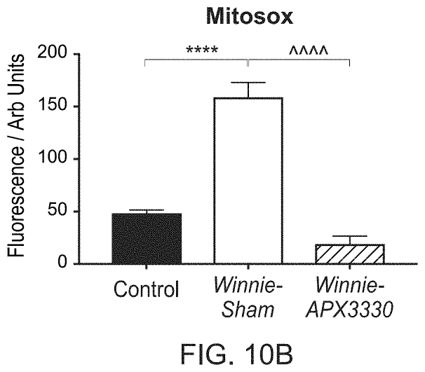

A fluorescent mitochondrial superoxide marker MITOSOX™ Red probed distal colon myenteric ganglion to evaluate levels of superoxide production from control C57BL/6, Winnie-sham treated, Winnie-APX3330 treated animals (FIG. 10A). Increased MitoSOX fluorescence was evident in the myenteric plexus from Winnie-sham treated mice (159.0±14%, P<0.0001, n=4) when compared to control mice (48.4±3.2%, n=4) (FIGS. 10A & 10B). Increased superoxide production in the distal colon myenteric plexus was alleviated in Winnie-APX3330 treated mice (19.2±7.5%, P<0.0001, n=4) compared to Winnie-sham treated mice (FIGS. 10A & 10B).

APX3330 Treatment Attenuated Cytoplasmic Translocation of HMGB1

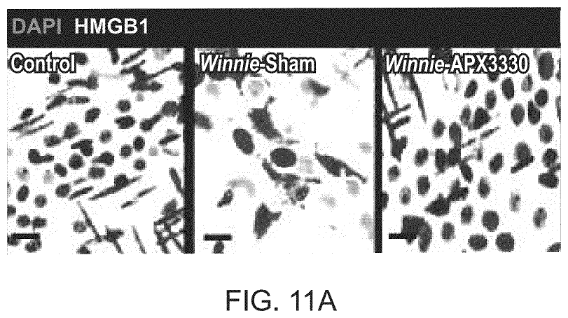

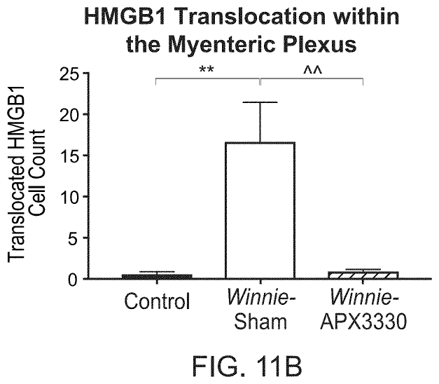

Inflammatory downstream pathways were assessed by an antibody against HMGB1 in the myenteric plexus of the inflamed colon (FIGS. 11A & 11B). HMGB1 translocation from nuclei to cytoplasm was measured by IR within LMMP preparations from control, Winnie-sham treated and Winnie-APX3330 treated mice (FIG. 11A). In Winnie-sham treated mice, a large number of cells quantified with HMGB1 translocation into the cytosol was present (16.6±4.8, P<0.01, n=4) when compared to control C57BL/6 mice (0.5±0.3, n=5) (FIGS. 11A & 11B). APX3330 treatment had attenuated cytoplasmic translocation of HMGB1 in the myenteric ganglia (0.9±0.3, P<0.01 n=4) compared to Winnie mice (FIGS. 11A & 11B).

APX3330 Treatment Reduced APE1 Overexpression in the Myenteric Ganglia

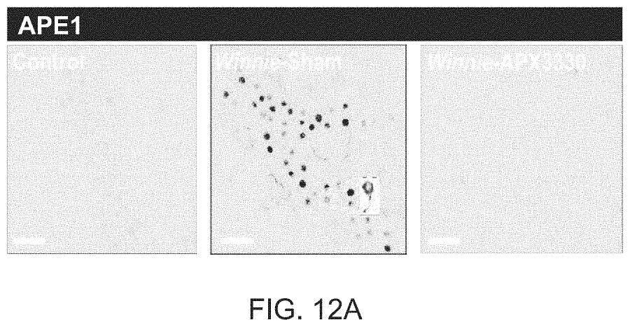

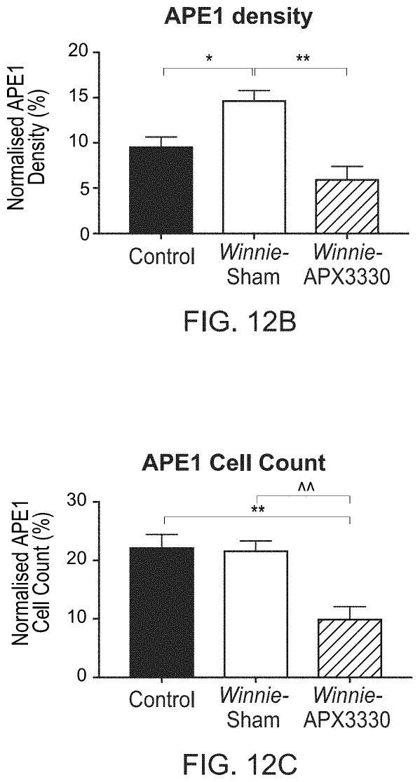

APE1 expression was determined by IR within the myenteric plexus in colon LMMP preparations from control C57BL/6, Winnie-sham treated and Winnie-APX3330 treated mice (FIGS. 12A-12C). It was observed that in Winnie-sham treated mice, APE1 was not only subjected to the nucleus, but also present in the cytosol (FIG. 12A). In Winnie-Sham treated mice there was a significant increase in APE1 intensity (14.7±1.0%, P<0.05, n=4) compared to control C57BL/6 mice (9.6±1.0%, n=5) (FIGS. 12A & 12B). On the contrary, Winnie-APX3330 treated mice had a reduction to overexpression of APE1 in the myenteric ganglia (6.0±1.4%, P<0.01, n=4) compared to Winnie-sham mice (FIGS. 12A & 12B). Further assessment quantified the number of APE1-IR cells in LMMP preparations (FIG. 12C). No changes in APE1 positive cells were found between control C57BL/6 mice (22.3±2.1%, n=5) and Winnie-sham treated mice (21.8±1.6%, P<0.01, n=4) (FIGS. 12A & 12C). However, the number of APE1-IR cells was significantly decreased in Winnie-APX3330 treated mice compared to both control C57BL/6 (P<0.01) and Winnie-sham treated mice (P<0.01) (FIGS. 12A & 12C).

APX3330 Treatment Repaired DNA Damage to Myenteric Neurons in the Distal Colon

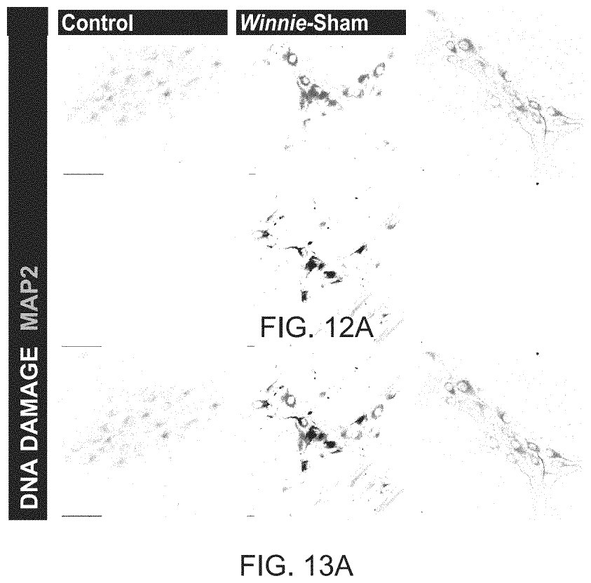

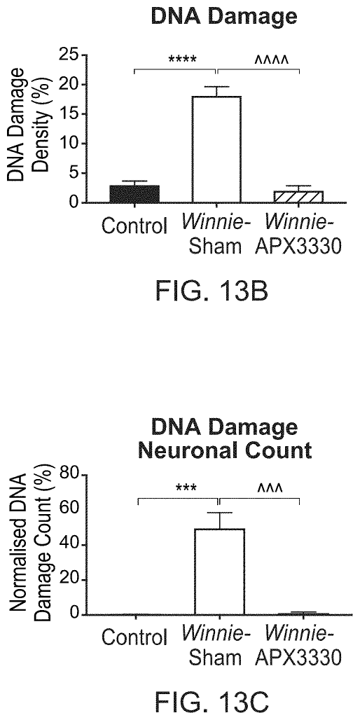

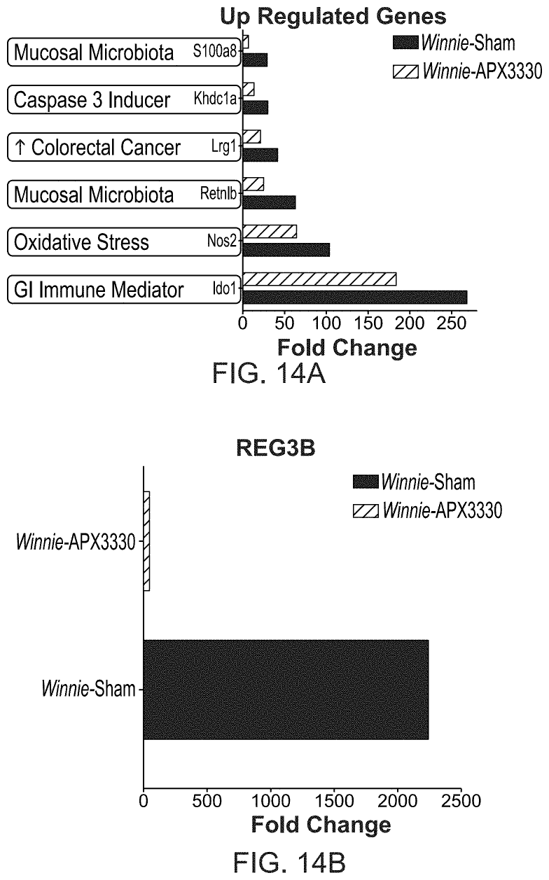

Co-immunolabelled myenteric neurons with, a pan neuronal marker, MAP2, and oxidative DNA damage marker, Oxo-8-dG were quantified (FIG. 13A). Overall, expression for Oxo-8dG-IR was significantly increased in Winnie-sham treated animals (18.1±1.6%, P<0.001, n=6) when compared to control C57BL/6 mice (2.97±0.7%, n=4) (FIGS. 13A & 13B). This expression subsided in Winnie-APX3330 treated mice (2.0±0.9%, P<0.001, n=6) when compared to Winnie-sham treated mice to the levels comparable to controls (FIGS. 13A & 13B). The number of myenteric neurons IR for Oxo-8dG was significantly increased in Winnie-sham treated mice (49±9.1, P<0.0001, n=6) when compared to control C57BL/6 mice (0.2±0.2, n=4) (FIGS. 13A & 13C). This was averted in Winnie-APX3330 treated mice (0.9±0.7, P<0.001, n=6) with a decrease in DNA damage expression in myenteric neurons in comparison to Winnie-sham treated mice (FIGS. 13A & 13C).

APX3330 Treatment Returned Up Regulated RNA Expressions Closer to Baseline

In order to profile changes in gene expression associated with inflammation, colorectal cancer susceptibility, microbiota alterations and oxidative stress PCR arrays of colon RNA were performed using pooled colon samples. Level of 5100 calcium-binding protein A8 (S100a8), KH homology domain-containing protein 1A (Khdc1a), Resistin-like beta (Retnlb), Leucine-rich alpha-2-glycoprotein (Lrg1) Nitric oxide synthase (Nos2) were analyzed.

In summary, this Example demonstrated the application of APX3330 treatment alleviates clinical symptoms and GI transit in the preclinical Winnie mice models of IBD. Hindering redox active domain of the APE1/Ref-1 molecule assumes restoration of antioxidant to oxidant balance by restoring cellular homeostasis, which coincides with improved clinical prospects of diarrhea and weight loss. Furthermore, elevated levels for ROS and RNS affiliates with a compromised immune response attributing to impaired GI functions.

Specific inhibition of the redox function of the APE1/Ref-1 molecule displayed preventative effects of oxidative stress induced enteric neuropathy and alleviated intestinal inflammation. Despite considerable evidence to support enteric dysregulation in IBD, the effect of oxidative stress and APE1/Ref-1 mechanistic role within the ENS had not been elucidated. Hence, the APE1/Ref-1 antagonist, APX3330, provided an opportunity in targeting specific redox mechanisms of the oxidative stress response associated with intestinal inflammation. Therefore, an understanding of APX3330's role in anti-inflammatory responses, enteric neuropathy, disease severity, GI function and symptoms of IBD was obtained.

Targeting the specific inhibition of APE1/Ref-1 redox pathways in a clinically relevant model led to a possible treatment of IBD and inflammation induced enteric neuropathy in human trials.

1. A method of treating inflammation and chronic pain in a subject suffering from functional gastrointestinal disease, the method comprising administering to the subject an effective amount of an apurinic/apyrimidinic endonuclease 1 redox factor 1 (APE1/Ref-1) inhibitor, pharmaceutically acceptable salts or pharmaceutically acceptable solvates thereof.

2. The method as set forth in claim 1, wherein the APE1/Ref-1 inhibitor has the formula: 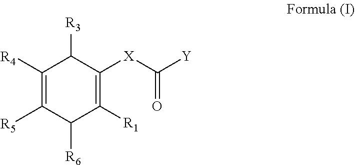
wherein R₁ is selected from the group consisting of alkyl, alkoxy, hydroxyl, and hydrogen; R₃ and R₆ are independently selected from the group consisting of a substituted or unsubstituted alkoxy, a substituted or unsubstituted aryl and an oxo; R₄ and R₅ are independently selected from the group consisting of an alkoxy and aryl, or both R₄ and R₅ taken together form a substituted or unsubstituted napthoquinone;
X is selected from the group consisting of CH═CR₂ and NCH, wherein R₂ is selected from the group consisting of C₁-C₁₀ alkyl and CF₃CH₂CH₂; and
Y is selected from the group consisting of N(Rz)R2 or NR\hat{ }OR\hat{ }, wherein each of Rz and R2 is independently selected from the group consisting of C₁-C₆ alkyl, heteroalkyl, cycloalkyl and cycloheteroalkyl, straight or branched chain or optionally substituted, or both Rz and R2 taken together with the attached nitrogen form an optionally substituted heterocycle; where each R\hat{ } is independently selected from the group consisting of hydrogen, alkyl, heteroalkyl, cyclohexyl, and cycloheteroalkyl, each of which is optionally substituted, or both R\hat{ } are taken together with the attached nitrogen and oxygen to form an optionally substituted heterocycle.

3. The method as set forth in claim 1, wherein the APE1/Ref-1 inhibitor is selected from an inhibitor set forth in Table 1.

4. The method as set forth in claim 1, wherein the APE1/Ref-1 inhibitor is selected from the group consisting of 3-[(5-(2,3-dimethoxy-6-methyl1,4-benzoquinoyl)]-2-nonyl-2-proprionic acid (APX3330), (2E)-2-[(3-methoxy-1,4-dioxo-1,4-dihydronapthalen-2-yl)methylidene]-N,N-dimethylpentanamide] (APX2007), [(2E)-2-[(3-methoxy-1,4-dioxo-1,4-dihydronaphthalen-2-yl)methylidene]-N,N-diethylpentanamide] (APX2009), (2E)-2-[(3-methoxy-1,4-dioxo-1,4-dihydronapthalen-2-yl)methylidene]-N-methoxypentanamide] (APX2014), (2E)-2-(3-methoxy-1,4-dioxo-1,4-dihydronaphthalen-2-yl)-N,N,2-trimethylprop-2-enamide (APX2032), pharmaceutically acceptable salts and pharmaceutically acceptable solvates thereof, and combinations thereof.

5. (canceled)

6. The method as set forth in claim 1 further comprising administering at least one additional therapeutic agent to the subject, wherein the additional therapeutic agent is selected from the group consisting of 5-aminosalicylic acid (5-ASA), corticosteroids, azathioprine, 6-mercaptopurine, methotrexate, cyclosporine, tacrolimus, anti-TNF drugs vedolizumab, natalizumab, ustekinumab, probiotics, antibiotics, anti-inflammatories, and combinations thereof.

7. (canceled)

8. The method as set forth in claim 1, wherein the subject is suffering from one or more of inflammatory bowel disease, Crohn disease (CD) and ulcerative colitis (UC), and indeterminate colitis (IC).

9. A method of reducing neuronal loss in a subject suffering from functional gastrointestinal disease, the method comprising the method comprising administering to the subject an effective amount of an apurinic/apyrimidinic endonuclease 1 redox factor 1 (APE1/Ref-1) inhibitor, pharmaceutically acceptable salts or pharmaceutically acceptable solvates thereof.

10. The method as set forth in claim 9, wherein the APE1/Ref-1 inhibitor has the formula: 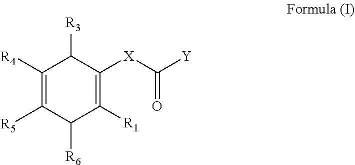
wherein R₁ is selected from the group consisting of alkyl, alkoxy, hydroxyl, and hydrogen; R₃ and R₆ are independently selected from the group consisting of a substituted or unsubstituted alkoxy, a substituted or unsubstituted aryl and an oxo; R₄ and R₅ are independently selected from the group consisting of an alkoxy and aryl, or both R₄ and R₅ taken together form a substituted or unsubstituted napthoquinone;
X is selected from the group consisting of CH═CR₂ and NCH, wherein R₂ is selected from the group consisting of C₁-C₁₀ alkyl and CF₃CH₂CH₂; and
Y is selected from the group consisting of N(Rz)R2 or NR\hat{ }OR\hat{ }, wherein each of Rz and R2 is independently selected from the group consisting of C₁-C₆ alkyl, heteroalkyl, cycloalkyl and cycloheteroalkyl, straight or branched chain or optionally substituted, or both Rz and R2 taken together with the attached nitrogen form an optionally substituted heterocycle; where each R\hat{ } is independently selected from the group consisting of hydrogen, alkyl, heteroalkyl, cyclohexyl, and cycloheteroalkyl, each of which is optionally substituted, or both R\hat{ } are taken together with the attached nitrogen and oxygen to form an optionally substituted heterocycle.

11. The method as set forth in claim 9, wherein the APE1/Ref-1 inhibitor is selected from an inhibitor set forth in Table 1.

12. The method as set forth in claim 9, wherein the APE1/Ref-1 inhibitor is selected from the group consisting of 3-[(5-(2,3-dimethoxy-6-methyl1,4-benzoquinoyl)]-2-nonyl-2-proprionic acid (APX3330), (2E)-2-[(3-methoxy-1,4-dioxo-1,4-dihydronapthalen-2-yl)methylidene]-N,N-dimethylpentanamide] (APX2007), [(2E)-2-[(3-methoxy-1,4-dioxo-1,4-dihydronaphthalen-2-yl)methylidene]-N,N-diethylpentanamide] (APX2009), (2E)-2-[(3-methoxy-1,4-dioxo-1,4-dihydronapthalen-2-yl)methylidene]-N-methoxypentanamide] (APX2014), (2E)-2-(3-methoxy-1,4-dioxo-1,4-dihydronaphthalen-2-yl)-N,N,2-trimethylprop-2-enamide (APX2032), pharmaceutically acceptable salts and pharmaceutically acceptable solvates thereof, and combinations thereof.

13. (canceled)

14. The method as set forth in claim 9 further comprising administering at least one additional therapeutic agent to the subject, wherein the additional therapeutic agent is selected from the group consisting of 5-aminosalicylic acid (5-ASA), corticosteroids, azathioprine, 6-mercaptopurine, methotrexate, cyclosporine, tacrolimus, anti-TNF drugs vedolizumab, natalizumab, ustekinumab, probiotics, antibiotics, anti-inflammatories, and combinations thereof.

15. (canceled)

16. A method of enhancing neurogenesis in a subject suffering from functional gastrointestinal disease, the method comprising administering to the subject an effective amount of an apurinic/apyrimidinic endonuclease 1 redox factor 1 (APE1/Ref-1) inhibitor, pharmaceutically acceptable salts or pharmaceutically acceptable solvates thereof, which selectively inhibits the amino terminal portion of APE1.

17. The method as set forth in claim 16, wherein the APE1/Ref-1 inhibitor has the formula: 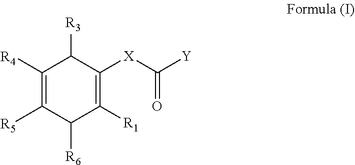
wherein R₁ is selected from the group consisting of alkyl, alkoxy, hydroxyl, and hydrogen; R₃ and R₆ are independently selected from the group consisting of a substituted or unsubstituted alkoxy, a substituted or unsubstituted aryl and an oxo; R₄ and R₅ are independently selected from the group consisting of an alkoxy and aryl, or both R₄ and R₅ taken together form a substituted or unsubstituted napthoquinone;
X is selected from the group consisting of CH═CR₂ and NCH, wherein R₂ is selected from the group consisting of C₁-C₁₀ alkyl and CF₃CH₂CH₂; and
Y is selected from the group consisting of N(Rz)R2 or NR\hat{ }OR\hat{ }, wherein each of Rz and R2 is independently selected from the group consisting of C1-C6 alkyl, heteroalkyl, cycloalkyl and cycloheteroalkyl, straight or branched chain or optionally substituted, or both Rz and R2 taken together with the attached nitrogen form an optionally substituted heterocycle; where each R\hat{ } is independently selected from the group consisting of hydrogen, alkyl, heteroalkyl, cyclohexyl, and cycloheteroalkyl, each of which is optionally substituted, or both R\hat{ } are taken together with the attached nitrogen and oxygen to form an optionally substituted heterocycle.

18. The method as set forth in claim 16, wherein the APE1/Ref-1 inhibitor is selected from an inhibitor set forth in Table 1.

19. The method as set forth in claim 16, wherein the APE1/Ref-1 inhibitor is selected from the group consisting of 3-[(5-(2,3-dimethoxy-6-methyl1,4-benzoquinoyl)]-2-nonyl-2-proprionic acid (APX3330), (2E)-2-[(3-methoxy-1,4-dioxo-1,4-dihydronapthalen-2-yl)methylidene]-N,N-dimethylpentanamide] (APX2007), [(2E)-2-[(3-methoxy-1,4-dioxo-1,4-dihydronaphthalen-2-yl)methylidene]-N,N-diethylpentanamide] (APX2009), (2E)-2-[(3-methoxy-1,4-dioxo-1,4-dihydronapthalen-2-yl)methylidene]-N-methoxypentanamide] (APX2014), (2E)-2-(3-methoxy-1,4-dioxo-1,4-dihydronaphthalen-2-yl)-N,N,2-trimethylprop-2-enamide (APX2032), pharmaceutically acceptable salts and pharmaceutically acceptable solvates thereof, and combinations thereof.

20. (canceled)

21. (canceled)

22. A method of myenteric and enteric neuronal protection in a subject in need thereof, the method comprising administering to the subject an effective amount of an apurinic/apyrimidinic endonuclease 1 redox factor 1 (APE1/Ref-1) inhibitor, pharmaceutically acceptable salts or pharmaceutically acceptable solvates thereof.

23. The method as set forth in claim 22, wherein the APE1/Ref-1 inhibitor has the formula: 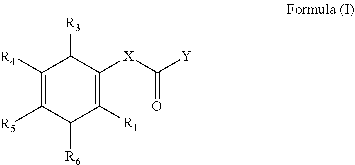
wherein R₁ is selected from the group consisting of alkyl, alkoxy, hydroxyl, and hydrogen; R₃ and R₆ are independently selected from the group consisting of a substituted or unsubstituted alkoxy, a substituted or unsubstituted aryl and an oxo; R₄ and R₅ are independently selected from the group consisting of an alkoxy and aryl, or both R₄ and R₅ taken together form a substituted or unsubstituted napthoquinone;
X is selected from the group consisting of CH═CR₂ and NCH, wherein R₂ is selected from the group consisting of C₁-C₁₀ alkyl and CF₃CH₂CH₂; and
Y is selected from the group consisting of N(Rz)R2 or NR\hat{ }OR\hat{ }, wherein each of Rz and R2 is independently selected from the group consisting of C₁-C₆ alkyl, heteroalkyl, cycloalkyl and cycloheteroalkyl, straight or branched chain or optionally substituted, or both Rz and R2 taken together with the attached nitrogen form an optionally substituted heterocycle; where each R\hat{ } is independently selected from the group consisting of hydrogen, alkyl, heteroalkyl, cyclohexyl, and cycloheteroalkyl, each of which is optionally substituted, or both R\hat{ } are taken together with the attached nitrogen and oxygen to form an optionally substituted heterocycle.

24. The method as set forth in claim 22, wherein the APE1/Ref-1 inhibitor is selected from an inhibitor set forth in Table 1.

25. The method as set forth in claim 22, wherein the APE1/Ref-1 inhibitor is selected from the group consisting of 3-[(5-(2,3-dimethoxy-6-methyl1,4-benzoquinoyl)]-2-nonyl-2-proprionic acid (APX3330), (2E)-2-[(3-methoxy-1,4-dioxo-1,4-dihydronapthalen-2-yl)methylidene]-N,N-dimethylpentanamide] (APX2007), [(2E)-2-[(3-methoxy-1,4-dioxo-1,4-dihydronaphthalen-2-yl)methylidene]-N,N-diethylpentanamide] (APX2009), (2E)-2-[(3-methoxy-1,4-dioxo-1,4-dihydronapthalen-2-yl)methylidene]-N-methoxypentanamide] (APX2014), (2E)-2-(3-methoxy-1,4-dioxo-1,4-dihydronaphthalen-2-yl)-N,N,2-trimethylprop-2-enamide (APX2032), pharmaceutically acceptable salts and pharmaceutically acceptable solvates thereof, and combinations thereof.

26. (canceled)

27. (canceled)

28. The method as set forth in claim 22, wherein the subject is suffering from one or more of inflammatory bowel disease, Crohn disease (CD) and ulcerative colitis (UC), and indeterminate colitis (IC).
